(19) United States
(12) Patent Application Publication
(10) Pub. No.: US 2016/0228444 A1
(43) Pub. Date: Aug. 11, 2016

(54) NUCLEOSIDE ANALOGS AND USES THEREOF

(71) Applicants: Ramot at Tel-Aviv University Ltd., Tel-Aviv (IL); Bar-Ilan University, Ramat-Gan (IL)

(72) Inventors: Ada Rephaeli, Herzlia (IL); Nataly Tarasenko, Petach-Tikva (IL); Abraham Nudelman, Rechovot (IL)

(21) Appl. No.: 15/002,711

(22) Filed: Jan. 21, 2016

Related U.S. Application Data
(60) Provisional application No. 62/106,333, filed on Jan. 22, 2015.

(51) Int. Cl. A61K 31/522; A61K 31/19; A61K 31/167; A61K 31/337; A61K 45/06; A61K 31/704

(52) U.S. Cl. CPC A61K 31/522; A61K 45/06; A61K 31/704; A61K 31/167; A61K 31/337; A61K 31/19

(57) ABSTRACT

Disclosed herein is a compound having general Formula I:

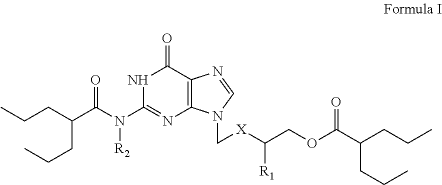

wherein X, R₁ and R₂ are as defined herein, for use in methods of treating an inflammatory disease or disorder, treating or preventing anthracycline-induced cardiomyopathy, downregulating interleukin-6, MCP-1, TNF-α, and/or TLR4, and/or upregulating interleukin-10.

### RELATED APPLICATION

This application claims the benefit of priority under 35 USC §119(e) of U.S. Provisional Patent Application No. 62/106,333 filed on Jan. 22, 2015, the contents of which are incorporated herein by reference in their entirety.

### FIELD AND BACKGROUND OF THE INVENTION

The present invention, in some embodiments thereof, relates to therapy, and more particularly, but not exclusively, to therapeutic uses of a nucleoside analog comprising a valproic acid moiety.

Histone deacetylases (HDACs; EC 3.5.1.98) are a class of enzymes that remove acetyl groups from lysine residues on a histone, allowing the histone to wrap the DNA more tightly, thereby resulting in a more condensed DNA structure which inhibits gene expression. HDACs also remove acetyl groups from proteins other than histones. 18 mammalian HDACs are currently known, which are classified into classes I, IIA, IIB, III and IV, based on sequence homology.

HDAC inhibitors include short chain fatty acids such as butyric acid, phenylbutyric acid and valproic acid; hydroxamic acids such as trichostatin A, vorinostat, panobinostat, abexinostat, pracinostat, resminostat, givinostat, quisinostat and belinostat; cyclic tetrapeptides such as romidepsin; and benzamides such as entinostat and mocetinostat.

International Patent Application having Publication No. WO 2008/120205 describes valproic acid derivatives of acyclovir (an antiviral drug), as well as their use in treating a proliferative disease or disorder.

The HDAC inhibitor AN446 (a derivative of valproic acid and acyclovir) has been reported to synergistically enhance the toxicity of doxorubicin towards tumors, while reducing the toxicity of doxorubicin towards non-cancerous cells [Tarasenko et al., Biochem Pharmacol 2014, 88:158-168].

Toll-like receptor 4 (TLR4) is expressed in triple negative breast carcinoma (TNBC), hepatocellular carcinoma (HCC), cervical, endometrial and ovarian cancers, and its expression has been reported to be associated with poor prognosis [Ma et al., PLoS ONE 2014, 9:e111639; Wang et al., Int J Mol Sci 2015, 16:22527-22540; Yang et al., PLoS ONE 2014, 9:e109980; Bergenfelz et al., Br J Cancer 2015, 113:1234-1243; Mehmeti et al., Breast Cancer Res 2015, 17:130; Ran, Cancer Res 2015, 75:2405-2410].

Additional background art includes Ageberg et al. [Am J Transl Res 2013, 5:170-183], Arce et al. [PLoS ONE 2006, 1:e98], Aune et al. [J Mol Cell Cardiol 2014, 72:138-145], Blaheta et al. [Br J Cancer 2007, 96:1699-1706], Bozic et al. [Elife 2013, 2:e00747], Candelaria et al. [Ann Oncol 2007, 18:1529-1538], Carraway & Gore [J Clin Oncol 2007, 25:1955-1956], Catalano et al. [J Endocrinol 2006, 191:465-472], Catalano et al. [Endocr Relat Cancer 2007, 14:839-845], Chavez-Blanco et al., [Cancer Cell Int 2006, 6:2], Chodurek et al. [Acta Pol Pharm 2012, 69:1298-1302], Cipro et al. [Oncol Rep 2012, 27:1219-1226], Dausukho et al. [Free Radic Biol Med 2007, 42:1818-1825], Dowdy et al. [Mol Cancer Ther 2006, 5:2767-2776], Engel et al., [J Cancer Res Clin Oncol 2006, 132:673-683], Fushida et al. [Onco Targets Ther 2015, 8:939-941], Hrebackova et al. [Curr Drug Targets 2010, 11:361-379], Jain et al. [Breast Cancer Res Treat 2012, 135:103-114], Jambalganiin et al. [Int Immunophamacol 2014, 20:181-187], Kee et al. [Kidney Blood Press Res 2013, 37:229-239], Kessler-Icekson et al. [Eur J Pharm Sci 2012, 45:592-599], Kim et al. [Int J Oncol 2009, 34:1353-1362], Lozano et al. [Neuropsychiatr Dis Treat 2015, 11:97-106], Mancini et al. [Cancers (Basel) 2014, 6:2187-2223], Marchion et al. [Mol Cancer Ther 2005, 4:1993-2000], Munster et al. [J Clin Oncol 2007, 25:1979-1985], More et al. [Mediators Inflamm 2013, 2013:952375], Munster et al. [Clin Cancer Res 2009, 15:2488-2496], Noguchi et al. [Endocrine J 2009, 56:245-249], Pusztai et al. [Cytokine 2004, 25:94-102], Rephaeli et al. [Br J Cancer 2007, 96:1667-1674], Rivera & Cianfrocca [Cancer Chemother Pharmacol 2015, 75:659-670], Robertson et al. [J Exp Ther Oncol 2013, 10:219-233], Rodriguez-Menendez et al. [Anticancer Res 2008, 28:335-342], Sanchez-Gonzalez et al. [Blood 2006, 108:1174-1182], Scherpereel et al. [Eur Respir J 2011, 37:129-135], Schuchmann et al. [Oncol Rep 2006, 15:227-230], Shakespear et al. [J Biol Chem 2013, 288:25362-25374], Sinn et al. [Neurobiol Dis 2007, 26:474-472], Suh et al. [J Neuroimmun Pharmacol 2010, 5:521-532], Tao et al. [Cell Signal 2014, 26:521-527], Tarasenko et al. [Invest New Drugs 2012, 30:130-143], Tarasenko et al. [PLoS ONE 2012, 7:e31393], Thurn et al. [Future Oncol 2011, 7:263-283], Valentini et al. [Cancer Biol Ther 2007, 6:185-191], van Beneden et al. [J Am Soc Nephrol 2011, 22:1863-1875], van Beneden et al. [Toxicol Appl Pharmacol 2013, 271:276-284], Vandermeers et al. [Clin Cancer Res 2009, 15:2818-2828], Wang et al. [Cardiovasc Res 2013, 98:56-63], Wang et al. [Oncol Lett 2013, 6:1492-1498], Wittenburg et al. [Cancer Chemother Pharmacol 2011, 67:83-92], Wittenburg et al. [Clin Cancer Res 2010, 16:4832-4842], Yokobori et al. [Mol Cancer Res 2014, 12:32-37], Zuco et al. [PLoS ONE 2011, 6:e29085] and International Patent Application having Publication No. WO 2005/120577.

### SUMMARY OF THE INVENTION

According to an aspect of some embodiments of the invention, there is provided a method of treating an inflammatory disease or disorder in a subject in need thereof, the method comprising administering to the subject a compound having general Formula I:

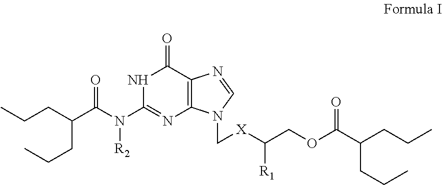

wherein:

X is selected from the group consisting of O and CH₂, or is absent;

R₁ is selected from the group consisting of H, —OR₃, —CH₂OR₃ and —CH₂CH₂OR₃; and

R₂ and R₃ are each independently hydrogen or valproyl,

thereby treating the inflammatory disease or disorder.

According to an aspect of some embodiments of the invention, there is provided a method of downregulating a protein selected from the group consisting of interleukin-6, MCP-1, TNF-α, and TLR4 (toll-like receptor 4) in a cell and/or subject, the method comprising contacting the cell and/or administering to the subject an effective amount of a compound having general Formula I:

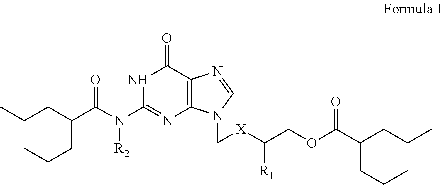

wherein:

X is selected from the group consisting of O and CH₂, or is absent;

R₁ is selected from the group consisting of H, —OR₃, —CH₂OR₃ and —CH₂CH₂OR₃; and

R₂ and R₃ are each independently hydrogen or valproyl,

thereby downregulating the protein.

According to an aspect of some embodiments of the invention, there is provided a method of upregulating interleukin-10 in a cell and/or subject, the method comprising contacting the cell and/or administering to the subject an effective amount of a compound a compound having general Formula I:

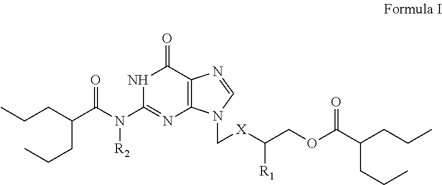

wherein:

X is selected from the group consisting of O and CH₂, or is absent;

R₁ is selected from the group consisting of H, —OR₃, —CH₂OR₃ and —CH₂CH₂OR₃; and

R₂ and R₃ are each independently hydrogen or valproyl, thereby of upregulating interleukin-10.

According to some embodiments of the invention, the disease or disorder is a disease or disorder in which downregulating a protein selected from the group consisting of interleukin-6, MCP-1, TNF-α, and TLR4 is beneficial.

According to some embodiments of the invention, the protein to be downregulated is TLR4.

According to some embodiments of the invention, the disease or disorder is selected from the group consisting of sepsis, cytokine storm, influenza, allergy to nickel, opioid tolerance and/or addiction, hyperalgesia, allodynia, and cancer associated with cancer cells which express TLR4.

According to some embodiments of the invention, the cancer is selected from the group consisting of triple negative breast carcinoma, gastric carcinoma, lung carcinoma, colon carcinoma, glioblastoma, hepatocellular carcinoma, cervical cancer, endometrial cancer and ovarian cancer.

According to some embodiments of the invention, the disease or disorder is the abovementioned cancer, and the subject is treated with a taxane drug.

According to some embodiments of the invention, the taxane drug is paclitaxel.

According to some embodiments of the invention, the disease or disorder is selected from the group consisting of diabetes, atherosclerosis, depression, schizophrenia, Alzheimer's disease, lupus erythematosus, Behcet's disease, multiple myeloma, prostate cancer, pancreatic cancer, rheumatoid arthritis, juvenile idiopathic arthritis, Castleman's disease, and enterovirus 71 infection.

According to some embodiments of the invention, the disease or disorder is a disease or disorder in which upregulating interleukin-10 is beneficial.

According to some embodiments of the invention, the disease or disorder is associated with fibrosis.

According to some embodiments of the invention, the fibrosis is not pulmonary fibrosis or hepatic cirrhosis.

According to some embodiments of the invention, the fibrosis is of an internal organ.

According to some embodiments of the invention, the internal organ is selected from the group consisting of heart and kidney.

According to some embodiments of the invention, the subject is treated with an anticancer therapy.

According to some embodiments of the invention, the subject is treated with an anticancer drug.

According to some embodiments of the invention, the anticancer drug is selected from the group consisting of an anthracycline and a platinum-based antineoplastic drug.

According to some embodiments of the invention, the fibrosis is cardiac fibrosis and the anticancer drug is an anthracycline.

According to some embodiments of the invention, the fibrosis is renal fibrosis and the anticancer drug is a platinum-based antineoplastic drug.

According to some embodiments of the invention, the platinum-based antineoplastic drug is cisplatin.

According to some embodiments of the invention, the method further comprises:

administering an anticancer therapy to the subject during a first time period,

following the first time period, determining whether the subject is afflicted with fibrosis, and

administering the compound to a subject determined to be afflicted with fibrosis during a second time period.

According to some embodiments of the invention, the method further comprises administering the anticancer therapy to the subject, and co-administering the compound with the anticancer therapy.

According to some embodiments of the invention, the disease or disorder is an inflammatory neurological disease or disorder.

According to some embodiments of the invention, the inflammatory neurological disease or disorder is selected from the group consisting of multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis (AML), stroke, an inflammatory retinal disease or disorder, an inflammatory ocular disease or disorder, optic neuritis, spongiform encephalopathy, migraine, headache, cluster headache, and stiff-man syndrome.

According to some embodiments of the invention, the inflammatory neurological disease or disorder is a traumatic brain injury.

According to some embodiments of the invention, X is O.

According to some embodiments of the invention, R₂ is hydrogen.

According to some embodiments of the invention, R₁ is selected from the group consisting of H and —CH₂OR₃.

According to some embodiments of the invention, R₁ is hydrogen.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D show in vitro anticancer activity of AN446. FIG. 1A is a bar graph showing a comparison of in vitro anticancer activity of AN446 and AN7 in various cancer cell lines (viability of adherent cells after 72 h was determined by a Hoechst viability assay whereas viability of suspension cells was measured using an MTT assay; average IC50 values were obtained from at least three independent-detailed titrations of cell survival vs. drug concentrations (±SE)). FIG. 1B is a bar graph showing inhibition of HDAC activity by AN446 and AN7 in U251 and Daudi cells (cells were incubated for 2 h with different concentrations of AN446 and AN7; average IC50 values of three independent HDAC inhibition experiments were calculated). FIG. 1C presents representative dot-plots (left) and a bar graph (right) showing results of FACS analysis of 4T1 and U251 MG cells treated with: 25 mM AN446 for 24 h; 200 nM Dox for 5 h and the combination of AN446+Dox where the cells were exposed to AN446 for 1 h, then 200 nM Dox was added for an additional 5 h, Dox was removed and AN446 was added back for additional 18 h incubation (at the end of the treatments, the cells were stained with annexin V-FITC and PI; height represents the number of cells and the shift to the right the increase in number of annexin V-FITC positive cells is shown on the left hand panel, and the average percent of dead cells (mean±SE) from three independent experiments is shown in the right hand panel). FIG. 1D presents representative histograms (upper panel) and a bar graph (lower panel) showing results of FACS analysis for measuring production of ROS in the cells treated for 6 h with: 25 mM AN446 for 6 h; 200 nM Dox for 5 h and the combination of AN446+Dox, where the cells were exposed to AN446 for 1 h, then 200 nM Dox was added for an additional 5 h. DCF-DA (10 mM) was added for 30 min (histograms show the increase in ROS production as a shift of the curve to the right; average of % DCF (+) cells, from three independent experiments, was calculated and is shown in lower panel; * indicates p<0.05 for treatments vs. untreated; # indicates p<0.05 for AN446+Dox vs. Dox; + indicates p<0.05 for AN446+Dox vs. AN446).

FIGS. 2A-2C show effects of AN446 (5-100 mM), Dox (5-60 nM) or their combination on the viability of U251 MG human glioma cells (3×10³/well; FIG. 2A) and 4T1 murine mammary cells (2×10³/well; FIG. 2B) seeded in 96 well-plates, incubated overnight and then treated with AN446, Dox or their combination at a molar ratio of 1:2000 (4T1) and 1:1000 (U251). After 72 h of treatment the viability of the cells was determined by a Hoechst assay and the average data of three independent experiments were calculated (drug concentration dependence plots were generated for each of the drugs alone and in combination using CompuSyn software; the fraction of affected cells (Fa) as function of Dox concentrations (nM) or AN446 concentrations (nM) as a single agent and AN446+Dox, are shown in the left-hand panel; combination indices (CIs) as function of Fa are shown in the right-hand panel). FIG. 2C is a table showing the median effect points.

FIGS. 3A-3F show the effect of AN7, AN446, Dox, and their combination on U251 human glioblastoma xenograft in 9-10-week-old male Hsd athymic FOXN mice inoculated subcutaneously with 5×10⁶ U251 cells. When tumor volume reached 50-100 mm³, the mice were assigned blind to the following treatments: saline ip (n=6) or po (n=6); 4 mg/kg ip Dox, once/week (n=10); 25 mg/kg po AN446 thrice a week (n=10); 50 mg/kg po AN7, 3 times/week (n=10); 25 mg/kg po AN446+4 mg/kg ip Dox (n=10); 50 mg/kg po AN7+4 mg/kg ip Dox (n=10). FIG. 3A is a Kaplan-Mier graph showing the percentage of failure-free mice (arrows on the x-axis indicate the time of Dox treatment). FIG. 3B is a graph showing body weight during the 25 days of treatment (#p<0.05 for combinations-treated vs. Dox-treated mice). FIG. 3C is graph showing tumor growth as function of time (volume mm³; * p<0.05 for drug-treated vs. vehicle-treated mice; ‡ p<0.05 for AN446 or AN7 in combination with Dox vs. AN446 or AN7 alone). FIG. 3D is a graph showing tumor weight at the termination point (g, mean±SE; * p<0.02 for drug-treated vs. vehicle-treated mice; # p<0.05 for combinations-treated vs. Dox-treated mice; ‡ p<0.05 for AN446 or AN7 in combination with Dox vs. AN446 or AN7 alone). FIG. 3E presents images and FIG. 3F is a bar graph showing evaluation of cell proliferation on tumor sections stained for Ki-67 (in FIG. 3E, bar=500 mm, the 5-fold magnified insert was taken from the area marked by the square; in FIG. 3F, counting of positive cells was performed on four different fields/section in three mice/group, results represent the average±SEM of 12 different fields, * p<0.05 for drug-treated vs. vehicle-treated mice, # p<0.05 for combinations-treated vs. Dox-treated; ‡ p<0.05 for AN446 or AN7 in combination with Dox vs. AN446 or AN7 alone).

FIGS. 4A-4F show modulation of HO-1 (FIGS. 4A and 4D), HO-1 mRNA (FIGS. 4B and 4E), bFGF (FIGS. 4A and 4D), bFGF mRNA (FIGS. 4B and 4E) and VEGF (FIGS. 4C and 4F) levels in vivo in tumor (FIGS. 4A-4C) and in heart (FIGS. 4D-4F) by the AN446, AN7, Dox or their combinations. Lysates of tumors or hearts (35 mg protein/well) were loaded on 12% SDS gel for the detection of HO-1, and on 15% SDS gel for the detection of bFGF, and were subjected to Western blot analyses using the specific antibodies (fold increase represents the ratio of band intensity (mean±SE)) of drug-treated to vehicle-treated, each normalized to actin signal, * p<0.05 for drug-treated vs. vehicle-treated mice; # p<0.05 for Dox-treated vs. combination-treated mice. In FIGS. 4A and 4B, representative immunoblots of HO-1 and bFGF in the tumors (FIG. 4A) and hearts (FIG. 4D) are shown below the graphs. In FIGS. 4B and 4E, HO-1 and bFGF mRNA expression was determined by real-time PCR. In FIGS. 4C and 4F, sections of tumors and hearts were immuno-stained for VEGF and the 5-fold magnified insert was taken from the area marked by the square; bar=500 mm.

FIGS. 5A and 5B present bar graphs and representative immunoblots showing the in vivo effect of AN-7, AN446, Dox and their combination on the expression of c-Myc protein and its phosphorylated form (p-c-Myc) in lysates of tumor (FIG. 5A) or heart (FIG. 5B) (lysates were loaded on 10% SDS gel (35 mg protein/well) for the detection of c-Myc or p-c-Myc (Thr 58) protein levels, and subjected to Western blot analyses using specific antibodies; fold increase represent the ratio of band intensity (mean±SE) of drug-treated to vehicle treated, each normalized to actin signal; * p<0.05 for drug-treated vs. vehicle-treated mice; # p<0.05 for Dox-treated vs. AN446- or AN7-treated mice).

FIG. 6 presents a table showing the average fold changes of target genes bFGF and collagen T1 in vivo (total RNA was extracted from tumors of nude mice bearing U251 tumors (n=4) subjected to qRT-PCR; data calculated using comparative quantification to the housekeeping gene GAPDH and normalized to the vehicle (2^{ΔΔCT}); ^{Δ} average of fold changes of target genes, which was calculated using comparative quantification to the housekeeping gene GAPDH (2^{ΔCT}); * p<0.05 for treated vs. vehicle-treated mice; ‡ p<0.05 for combination-treated vs. Dox-treated mice).

FIGS. 7A-7C present bar graphs (FIGS. 7A and 7B) showing the effect of AN446 (at a concentration of 0, 20 or 40 μM) on THP1 cells treated with 1 μg/ml lipopolysaccharide (LPS) compared to untreated cells (untr), as manifested by the percentage of cells expressing TLR4 (FIG. 7A) and by concentrations of IL-6, IL-10 and TNF-α in the supernatant of the cells (FIG. 7B); and a table (FIG. 7C) showing gene expression of MCP-1, TNF-α and TLR4 in MDA-231 cells, as quantified by qRT-PCR, following treatment with AN446, paclitaxel (PXL) and their combination.

FIGS. 8A-8C present images of tumor, heart and kidney tissue (FIG. 8A) from Hsd:athymic nude-Foxn mice inoculated subcutaneously in the flanks with 5×10⁶ U251 cells and treated with vehicle (saline), doxorubicin (Dox; intraperitoneally administered), AN446 (per os) or AN446 and doxorubicin, the tissue being stained for fibrous collagen with 0.1% Picrosirius Red and 0.2% Fast Green; representative images of heart sections (FIG. 8B) immunostained for Ki67 (for staining proliferating cells), vimentin (for staining fibroblasts) and DAPI, for identifying proliferating fibroblasts; and a table (FIG. 8C) showing levels of serum TNF-α (measured by a ELISA kit and amounts (relative to vehicle treatment) of Ki67-positive and vimentin-positive cells in heart sections of mice treated with AN446 and/or doxorubicin (Dox).

FIG. 9 presents a bar graph and a table showing the percentage of dead cells among U251 glioblastoma cells treated for 24, 48 or 72 hours with 2 μm entinostat, 1 nm romidepsin, 1 μm SAHA, 3 mM valproic acid (VPA) or 20 μm AN446 (valprostat), as well as among untreated control glioblastoma cells.

FIG. 10 presents a bar graph and a table showing the percentage of dead cells among H9C2 cardiomyoblasts treated for 24, 48 or 72 hours with 2 μm entinostat, 1 nm romidepsin, 1 μm SAHA, 3 mM valproic acid (VPA) or 20 μm AN446 (valprostat), as well as among untreated control cardiomyoblasts.

FIG. 11 presents a bar graph showing therapeutic indexes of entinostat, romidepsin, SAHA, valproic acid (VPA) and AN446 (valprostat) based on the ratio of cancer cell mortality (as shown in FIG. 9) to cardiomyoblast mortality (as shown in FIG. 10) after 24, 48 and 72 hours.

FIG. 12 presents images of Western blots showing levels of acetylated H3 histone (Acet H3) and total H3 histone (Total H3) in U251 glioblastoma cells and H9C2 cardiomyoblasts treated with entinostat, romidepsin, SAHA, valproic acid (VPA) and AN446 (valprostat), and in untreated (Unt) cells, and a bar graph and table showing the ratio of the level of acetylated H3 histone treated cells to the level of acetylated H3 histone in untreated cells (acetylation ratio) for entinostat, romidepsin, SAHA, valproic acid (VPA) and AN446 (valprostat) in U251 (dark bars) and H9C2 (light bars) cells (levels of acetylated and total H3 histone were quantified according to optical density, and levels of acetylated H3 histone were normalized to the levels of total H3 histone, which serve as a loading control).

FIGS. 13A and 13B present images of Western blots showing levels of phosphorylated H2AX (p-H2AX) in U251 cells (FIG. 13A) and H9C2 cells (FIG. 13B) treated for 24 or 48 hours with entinostat, romidepsin, SAHA, valproic acid (VPA) and AN446 (valprostat), and in untreated cells (left-most column), and a bar graph showing the ratio of p-H2AX levels in U251 (FIG. 13A) and H9C2 (FIG. 13B) cells treated with entinostat, romidepsin, SAHA, valproic acid (VPA) and AN446 (valprostat) to p-H2AX levels in untreated cells (actin levels serve as a control for the Western blot).

FIGS. 14A and 14B present images of Western blots showing levels of Rad51 in U251 cells (FIG. 14A) and H9C2 cells (FIG. 14B) treated for 24 or 48 hours with entinostat, romidepsin, SAHA, valproic acid (VPA) and AN446 (valprostat), and in untreated cells, and a bar graph showing the ratio of Rad51 levels in U251 (FIG. 14A) and H9C2 (FIG. 14B) cells treated with entinostat, romidepsin, SAHA, valproic acid (VPA) and AN446 (valprostat) to Rad51 levels in untreated cells (actin levels serve as a control for the Western blot).

FIG. 15 presents a bar graph showing TLR4 levels in MDA-231 cells treated for 24 hours with 2 μm entinostat, 1 nm romidepsin, 1 μm SAHA, 3 mM valproic acid (VPA) or 20 μm AN446 (valprostat), as a percentage of TLR4 levels in untreated control cells (results for each treatment group represent average of 3 independent experiments; * indicates p<0.05 vs. untreated cells, ** indicates p<0.05 vs. VPA-treated cells and untreated cells).

FIG. 16 presents a bar graph showing IL-6 concentration in cell culture of human normal astrocytes after incubation for 24 hours, for untreated cells (Untreated), and for cells incubated with 1 μg/ml lipopolysaccharide with (valprostat) and without (Untr+LPS) 24 hour pre-treatment with 20 μM valprostat (AN446).

### DESCRIPTION OF SPECIFIC EMBODIMENTS OF THE INVENTION

The present invention, in some embodiments thereof, relates to therapy, and more particularly, but not exclusively, to therapeutic uses of a nucleoside analog comprising a valproic acid moiety.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that a valproate/valproamide derivative of acyclovir, ameliorates cardiac and renal fibrosis in a surprisingly potent manner, and exhibits anti-inflammatory properties in various cell types.

Furthermore, as shown in the Examples section herein, a combination of an exemplary valproate/valproamide derivative of acyclovir (AN446) and an exemplary anticancer agent (doxorubicin or paclitaxel), compared to treatment with the anticancer agent alone, resulted in a response that was cell-type-specific, for example, causing a significant potentiation of ROS release and increased mortality of cancer cells, while significantly decreasing ROS and mortality induced by the anticancer agent in non-cancerous cells.

Embodiments of the present invention therefore relate to the use of valproate/valproamide derivatives of acyclovir in the treatment or prevention of inflammatory diseases and disorders, and of fibrosis in particular. Embodiments of the present invention therefore relate to the use of valproate/valproamide derivatives of acyclovir in protecting against adverse effects associated with toxicity of anticancer therapy to non-cancerous tissue (e.g., cardiomyopathy associated with anthracycline administration), while enhancing the toxicity of anticancer therapy to cancerous cells.

According to an aspect of some embodiments of the invention, there is provided a method of treating an inflammatory disease or disorder in a subject in need thereof, the method comprising administering to the subject a compound having general Formula I:

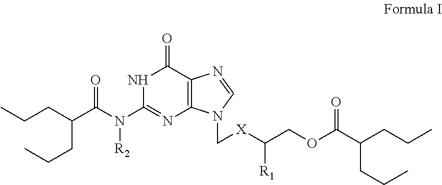

wherein:

X is O or CH₂, or is absent;

R₁ is H, —OR₃, —CH₂OR₃ or —CH₂CH₂OR₃; and

R₂ and R₃ are each independently hydrogen or valproyl,

thereby treating the inflammatory disease or disorder.

According to an aspect of some embodiments of the invention, there is provided a compound having general Formula I:

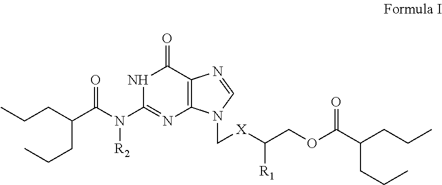

wherein:

X is O or CH₂, or is absent;

R₁ is H, —OR₃, —CH₂OR₃ or —CH₂CH₂OR₃; and

R₂ and R₃ are each independently hydrogen or valproyl,

for use in treating an inflammatory disease or disorder in a subject in need thereof.

As used herein, the phrase “treating” and “treatment” include abrogating, substantially inhibiting, slowing or reversing the progression of a condition, substantially ameliorating clinical or aesthetical symptoms of a condition or substantially preventing the appearance of clinical or aesthetical symptoms of a condition.

Representative examples of inflammatory diseases or disorders, treatable by the embodiments of the present invention, include, for example, idiopathic inflammatory diseases or disorders, chronic inflammatory diseases or disorders, acute inflammatory diseases or disorders, autoimmune diseases or disorders, infectious diseases or disorders, inflammatory transplantation-related diseases or disorders, inflammatory degenerative diseases or disorders, diseases or disorders associated with a hypersensitivity, inflammatory cardiovascular diseases or disorders, inflammatory cerebrovascular diseases or disorders, peripheral vascular diseases or disorders, inflammatory glandular diseases or disorders, inflammatory gastrointestinal diseases or disorders, inflammatory cutaneous diseases or disorders, inflammatory hepatic diseases or disorders, inflammatory neurological diseases or disorders, inflammatory musculo-skeletal diseases or disorders, inflammatory renal diseases or disorders, inflammatory reproductive diseases or disorders, inflammatory tumors (e.g., non-malignant tumors), inflammatory systemic diseases or disorders, inflammatory connective tissue diseases or disorders, necrosis, inflammatory implant-related diseases or disorders, inflammatory aging processes, immunodeficiency diseases or disorders, proliferative diseases and disorders and inflammatory pulmonary diseases or disorders, as is detailed herein.

In some embodiments of any one of the embodiments described herein, the inflammatory disease or disorder is not a cancer.

In some embodiments of any one of the embodiments described herein, the inflammatory disease or disorder is not a proliferative disease or disorder.

In some embodiments of any one of the embodiments described herein, the inflammatory disease or disorder is not cancer, stenosis, restenosis, in-stent stenosis, vascular graft restenosis, arthritis, rheumatoid arthritis, diabetic retinopathy, angiogenesis, pulmonary fibrosis, hepatic cirrhosis, atherosclerosis, glomerulonephritis, diabetic nephropathy, thrombic microangiopathy syndromes or transplant rejection.

In embodiments described herein wherein the subject has cancer and/or another a proliferative disease or disorder, the compound having general Formula I (according to any of the respective embodiments described herein) is optionally administered to treat an inflammatory condition associated with the cancer (e.g., fibrosis induced by an anticancer agent, resistance to an anticancer agent), whereas another agent (e.g., an anticancer agent as described herein) is preferably administered in order to treat cancer and/or proliferative disease or disorder.

Non-limiting examples of hypersensitivities include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

Non-limiting examples of inflammatory cardiovascular disease or disorder include occlusive diseases or disorders, atherosclerosis, a cardiac valvular disease, stenosis, restenosis, in-stent-stenosis, myocardial infarction, coronary arterial disease, acute coronary syndromes, congestive heart failure, angina pectoris, myocardial ischemia, thrombosis, Wegener's granulomatosis, Takayasu's arteritis, Kawasaki syndrome, anti-factor VIII autoimmune disease or disorder, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease or disorder, and anti-helper T lymphocyte autoimmunity.

Stenosis is an occlusive disease of the vasculature, commonly caused by atheromatous plaque and enhanced platelet activity, most critically affecting the coronary vasculature.

Restenosis is the progressive re-occlusion often following reduction of occlusions in stenotic vasculature. In cases where patency of the vasculature requires the mechanical support of a stent, in-stent-stenosis may occur, re-occluding the treated vessel.

Non-limiting examples of cerebrovascular diseases or disorders include stroke, cerebrovascular inflammation, cerebral hemorrhage and vertebral arterial insufficiency.

Non-limiting examples of peripheral vascular diseases or disorders include gangrene, diabetic vasculopathy, ischemic bowel disease, thrombosis, diabetic retinopathy and diabetic nephropathy.

Non-limiting examples of autoimmune diseases or disorders include all of the diseases caused by an immune response such as an autoantibody or cell-mediated immunity to an autoantigen and the like. Representative examples are chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides and heparin induced thrombocytopenia.

Non-limiting examples of inflammatory glandular diseases or disorders include pancreatic diseases or disorders, Type I diabetes, thyroid diseases or disorders, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Non-limiting examples of inflammatory gastrointestinal diseases or disorders include colitis, ileitis, Crohn's disease, chronic inflammatory intestinal disease, inflammatory bowel syndrome, chronic inflammatory bowel disease, celiac disease, ulcerative colitis, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer.

Non-limiting examples of inflammatory cutaneous diseases or disorders include acne, an autoimmune bullous skin disease, pemphigus vulgaris, bullous pemphigoid, pemphigus foliaceus, contact dermatitis and drug eruption.

Non-limiting examples of inflammatory hepatic diseases or disorders include autoimmune hepatitis, hepatic cirrhosis, and biliary cirrhosis.

Non-limiting examples of inflammatory neurological diseases or disorders include traumatic brain injury, multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis (AML), stroke, an inflammatory retinal disease or disorder, an inflammatory ocular disease or disorder, optic neuritis, spongiform encephalopathy, migraine, headache, cluster headache, and stiff-man syndrome.

Non-limiting examples of inflammatory connective tissue diseases or disorders include autoimmune myositis, primary Sjogren's syndrome, smooth muscle autoimmune disease or disorder, myositis, tendinitis, a ligament inflammation, chondritis, a joint inflammation, a synovial inflammation, carpal tunnel syndrome, arthritis, rheumatoid arthritis, osteoarthritis, ankylosing spondylitis, a skeletal inflammation, an autoimmune ear disease or disorder, and an autoimmune disease or disorder of the inner ear.

Non-limiting examples of inflammatory renal diseases or disorders include autoimmune interstitial nephritis and/or renal cancer.

Non-limiting examples of inflammatory reproductive diseases or disorders include repeated fetal loss, ovarian cyst, or a menstruation associated disease or disorder.

Non-limiting examples of inflammatory systemic diseases or disorders include systemic lupus erythematosus, systemic sclerosis, septic shock, toxic shock syndrome, and cachexia.

Non-limiting examples of infectious disease or disorder include chronic infectious diseases or disorders, a subacute infectious disease or disorder, an acute infectious disease or disorder, a viral disease or disorder, a bacterial disease or disorder, a protozoan disease or disorder, a parasitic disease or disorder, a fungal disease or disorder, a mycoplasma disease or disorder, gangrene, sepsis, a prion disease or disorder, influenza, tuberculosis, malaria, acquired immunodeficiency syndrome, and severe acute respiratory syndrome.

Non-limiting examples of inflammatory transplantation-related diseases or disorders include graft rejection, chronic graft rejection, subacute graft rejection, acute graft rejection hyperacute graft rejection, and graft versus host disease or disorder. Exemplary implants include a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

Non-limiting examples of inflammatory tumors include a malignant tumor, a benign tumor, a solid tumor, a metastatic tumor and a non-solid tumor.

Non-limiting examples of inflammatory pulmonary diseases or disorders include asthma, allergic asthma, emphysema, chronic obstructive pulmonary disease or disorder, pulmonary fibrosis, sarcoidosis and bronchitis.

In some embodiments of any one of the embodiments described herein, the compound or method is for downregulating interleukin-6, MCP-1, TNF-α, and/or TLR4 (toll-like receptor 4) in a cell (e.g., in vivo or ex vivo) and/or subject.

In some embodiments of any one of the embodiments described herein, the inflammatory disease or disorder is a disease or disorder in which downregulating interleukin-6, MCP-1, TNF-α, and/or TLR4 is beneficial.

In some embodiments of any one of the embodiments described herein, the compound or method is for upregulating interleukin-10 in a cell (e.g., in vivo or ex vivo) and/or subject.

In some embodiments of any one of the embodiments described herein, the inflammatory disease or disorder is a disease or disorder in which upregulating interleukin-10 is beneficial.

According to an aspect of some embodiments of the present invention there is provided a method of downregulating interleukin-6 (IL-6), MCP-1, TNF-α, and/or TLR4 (toll-like receptor 4) in a cell (e.g., in vivo or ex vivo) and/or subject, the method comprising contacting the cell and/or administering to the subject an effective amount of a compound of general Formula I as described herein. In some embodiments, the compound or method is for downregulating TLR4.

According to an aspect of some embodiments of the present invention there is provided a method of treating a disease or disorder in which downregulating interleukin-6 (IL-6), MCP-1, TNF-α, and/or TLR4 is beneficial, the method comprising administering to a subject in need thereof an effective amount of a compound of general Formula I as described herein. In some embodiments, the disease or disorder is a disease or disorder in which downregulating TLR4 is beneficial.

According to an aspect of some embodiments of the present invention there is provided a method of upregulating interleukin-10 in a cell (e.g., in vivo or ex vivo) and/or subject, the method comprising contacting the cell and/or administering to the subject an effective amount of a compound of general Formula I as described herein.

According to an aspect of some embodiments of the present invention there is provided a method of treating a disease or disorder in which upregulating interleukin-10 is beneficial, the method comprising administering to a subject in need thereof an effective amount of a compound of general Formula I as described herein.

Examples of diseases or disorders in which downregulating TLR4 may be beneficial include, without limitation, sepsis (e.g., septic shock), cytokine storm (e.g., cytokine storm associated with graft versus host disease, acute respiratory distress syndrome, sepsis, Ebola, influenza, smallpox, and/or systemic inflammatory response syndrome), influenza (e.g., avian influenza, swine flu, and any influenza strain identified as a potential cause of cytokine storms), allergy to nickel (e.g., contact allergy), side effects of opioid use (e.g., opioid tolerance, addiction and/or abuse, respiratory depression), hyperalgesia (e.g., hyperalgesia associated with opioid use), allodynia (e.g., allodynia associated with opioid use), and cancers associated with cancer cells which express (and optionally overexpress) TLR4.

Examples of cancers associated with cancer cells which express TLR4 include, without limitation, triple negative breast carcinoma, gastric carcinoma, lung carcinoma, colon carcinoma, glioblastoma, hepatocellular carcinoma, cervical cancer, endometrial cancer and ovarian cancer. It is to be understood that not all variants of the aforementioned cancers necessarily express TLR4. However, it is well within the abilities of the skilled person to determine whether a cancer in a given patient expresses TLR4.

In some embodiments of any of the embodiments described herein relating to treatment of a cancer, the subject is treated with an anticancer therapy, for example, an anticancer drug. In some such embodiments, the treatment comprises co-administration of a compound of general Formula I as described herein and an anticancer drug (e.g., an anticancer drug according to any of the respective embodiments described herein).

In some embodiment of any of the embodiments described herein relating to a disease or disorder in which downregulating IL-6, MCP-1, TNF-α, and/or TLR4 is beneficial, the subject is treated with a taxane drug. In some such embodiments, the disease or disorder is a taxane-induced inflammatory process.

In some embodiment of any of the embodiments described herein relating to a cancer associated with cancer cells which express TLR4, the subject is treated with a taxane drug.

Examples of taxane drugs include, without limitation, paclitaxel, docetaxel and cabazitaxel. Paclitaxel is an exemplary taxane drug.

As exemplified herein, paclitaxel interacted synergistically with a compound of general Formula I in cancer cells expressing TLR4.

It is expected that during the life of a patent maturing from this application many relevant taxanes will be developed and the scope of the term “taxane” is intended to include all such new technologies a priori.

Examples of diseases or disorders in which downregulating IL-6 may be beneficial include, without limitation, diabetes, atherosclerosis, depression, schizophrenia, Alzheimer's disease, lupus erythematosus (e.g., systemic lupus erythematosus), Behcet's disease, cancer associated with elevated IL-6 blood levels (e.g., multiple myeloma, prostate cancer, pancreatic cancer), arthritis (e.g., rheumatoid arthritis, systemic juvenile idiopathic arthritis), Castleman's disease, and enterovirus 71 infection (e.g., enterovirus 71 encephalitis and/or hand, foot and mouth disease).

Examples of diseases or disorders in which downregulating MCP-1 may be beneficial include, without limitation, psoriasis, arthritis (e.g., rheumatoid arthritis), atherosclerosis, glomerulonephritis, neuroinflammatory diseases and disorders (e.g., epilepsy, brain ischemia, Alzheimer's disease, multiple sclerosis, traumatic brain injury) and diabetes.

Examples of diseases or disorders in which downregulating TNF-α may be beneficial include, without limitation, psoriasis (e.g., plaque psoriasis), arthritis (e.g., rheumatoid arthritis, juvenile rheumatoid arthritis, psoriatic arthritis), ankylosing spondylitis, inflammatory bowel disease (e.g., Crohn's disease, ulcerative colitis), hidradenitis suppurativa, Behcet's disease, relapsing polychondritis, and asthma (e.g., refractory asthma).

Examples of diseases or disorders in which upregulating interleukin-10 may be beneficial include, without limitation, psoriasis, arthritis (e.g., rheumatoid arthritis), multiple sclerosis and inflammatory bowel disease (e.g., Crohn's disease).

In some embodiments of any of the embodiments described herein relating to an inflammatory disease or disorder, the disease or disorder is associated with fibrosis.

According to an aspect of some embodiments of the present invention there is provided a method of treating fibrosis in a subject in need thereof, the method comprising administering to the subject in need thereof an effective amount of a compound of general Formula I as described herein.

According to an aspect of some embodiments of the invention, there is provided a compound having general Formula I (according to any of the respective embodiments described herein), for use in the treatment of fibrosis in a subject in need thereof.

As used herein, the term “fibrosis” refers to formation of excess fibrous connective tissue in an organ or tissue. The term “fibrosis” includes, without limitation, scarring, in which the formation of fibrous tissue is in response to an injury, and fibromas, in which the fibrosis arises from a single cell line.

Examples of fibrosis include, without limitation, pulmonary fibrosis, liver cirrhosis, fibrosis associated with an anticancer therapy (e.g., radiation or anticancer drug), endomyocardial fibrosis, fibrosis associated with myocardial infarction, atrial fibrosis, mediastinal fibrosis, myelofibrosis, retroperitoneal fibrosis, progressive massive fibrosis, nephrogenic systemic fibrosis (a skin fibrosis), Crohn's disease, keloid fibrosis (a skin fibrosis), arthrofibrosis, Peyronie's disease, Dupuytren's contracture, and fibrosis associated with adhesive capsulitis.

Fibrosis represents a massive health care burden worldwide. Fibrogenesis commonly results in organ dysfunction and eventually in organ failure. Despite the well-recognized link between fibrosis and organ dysfunction, the therapeutic repertoire for the treatment of tissue fibrosis is severely limited and organ transplantation is currently the only effective treatment in end-stage fibrotic disease. However, organ transplantation has several disadvantages; therefore, there is an urgent need to develop new and effective anti-fibrotic therapies.

In some embodiments of any of the embodiments described herein relating to fibrosis, the fibrosis is not malignant.

In some embodiments of any of the embodiments described herein relating to fibrosis, the fibrosis is not pulmonary fibrosis or hepatic cirrhosis.

In some embodiments of any of the embodiments described herein relating to fibrosis, the fibrosis is of an internal organ (e.g., not fibrosis in skin). In some embodiments, the internal organ is heart and/or kidney (e.g., the fibrosis is cardiac fibrosis and/or renal fibrosis).

In some embodiments of any of the embodiments described herein relating to fibrosis, the fibrosis is associated with an anticancer therapy.

Herein, the phrase “anticancer therapy” encompasses administration of an anticancer drug as well as administration of at least one dose of radiation.

The appearance of anticancer therapy-induced fibrosis in vital organs is a severe, frequently encountered, side-effect of chemotherapy and radiation.

In some embodiments of any of the embodiments described herein, the anticancer therapy comprises administration of an anticancer drug to the subject.

Herein, the phrase “anticancer drug” encompasses any compound (excluding a compound having general Formula I) known in the art as beneficial in the treatment of at least one type of cancer.

Examples of anticancer drugs which may be used according to any of the respective embodiments described herein, include, without limitation, acivicin, aclarubicin, acodazole, acronine, adozelesin, aldesleukin, altretamine, ambomycin, ametantrone, aminoglutethimide, amsacrine, anastrozole, anthramycin, asparaginase, asperlin, azacitidine, azetepa, azotomycin, batimastat, benzodepa, bicalutamide, bisantrene, bisnafide, bizelesin, bleomycin, brequinar, bropirimine, busulfan, cactinomycin, calusterone, caracemide, carbetimer, carboplatin, carmustine, carubicin, carzelesin, cedefingol, chlorambucil, cirolemycin, cisplatin, cladribine, crisnatol, cyclophosphamide, cytarabine, dacarbazine, dactinomycin, daunorubicin, decitabine, dexormaplatin, dezaguanine, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, gemcitabine, hydroxyurea, idarubicin, ifosfamide, ilmofosine, interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, paclitaxel, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, zorubicin, and any pharmaceutically acceptable salts thereof.

In some embodiments of any of the embodiments described herein, the anticancer drug is an anthracycline or a platinum-based antineoplastic drug.

Herein, the term “anthracycline” refers to a therapeutically active agent used to prevent cell proliferation, which comprises a 9,10-anthraquinone moiety. Examples of anthracyclines include, without limitation, aclarubicin, carubicin, doxorubicin, daunorubicin, epirubicin, esorubicin idarubicin, valrubicin, zorubicin, mitoxantrone and losoxantrone.

It is expected that during the life of a patent maturing from this application many relevant anthracyclines and other anticancer drugs will be developed and the scope of the terms “anticancer drug” and “anthracycline” is intended to include all such new technologies a priori.

In some embodiments of any of the embodiments described herein relating to an anthracycline, the anthracycline comprises an additional ring fused to the anthraquinone moiety (e.g., as in doxorubicin, daunorubicin, epirubicin, idarubicin, valrubicin).

Herein, the term “platinum-based antineoplastic drug” refers to a therapeutically active agent used to prevent cell proliferation, which comprises at least one platinum atom. Examples of platinum-based antineoplastic drugs include, without limitation, cisplatin, carboplatin, dexormaplatin, enloplatin, iproplatin, lipoplatin, nedaplatin, ormaplatin, oxaliplatin, satraplatin, picoplatin, spiroplatin, triplatin and zeniplatin.

In some embodiments of any of the embodiments described herein relating to a platinum-based antineoplastic drug, the platinum-based antineoplastic drug is cisplatin.

It is expected that during the life of a patent maturing from this application many relevant platinum-based antineoplastic drugs will be developed and the scope of the term “platinum-based antineoplastic drug” is intended to include all such new technologies a priori.

Without being bound by any particular theory, in view of the data presented in the Examples section herein, it is believed that major contributors to anthracycline- and platinum-based antineoplastic drug-induced toxicities are the formation of reactive oxygen species (ROS) and inflammation that culminate in fibrosis leading to tissue-damage and loss of function.

It is to be appreciated that metastatic breast cancer (BC) patients are commonly treated by standard protocols that include doxorubicin and cisplatin, and gastric carcinoma (GC) patients are commonly treated with cisplatin. Both agents induce fibrosis. These oncological populations are generally regarded as being at high risk of fibrosis. Survival of metastatic BC patient varies greatly, however it is ˜20%. GC represents the fourth most common type of cancer, but the second leading cause of cancer death worldwide.

In some embodiments of any of the embodiments described herein, the anticancer drug is an anthracycline and the fibrosis is cardiac fibrosis and/or renal fibrosis. In some such embodiments, the fibrosis is cardiac fibrosis.

In some embodiments of any of the embodiments described herein, the anticancer drug is a platinum-based antineoplastic drug and the fibrosis is renal fibrosis. In some such embodiments, the platinum-based antineoplastic drug is cisplatin.

According to an aspect of some embodiments of the invention, there is provided a method of treating or preventing anthracycline-induced cardiomyopathy in a subject in need thereof, the method comprising administering to the subject a compound having general Formula I (according to any of the respective embodiments described herein).

According to an aspect of some embodiments of the invention, there is provided a compound having general Formula I (according to any of the respective embodiments described herein), for use in treating or preventing anthracycline-induced cardiomyopathy in a subject in need thereof.

Herein, the term “anthracycline-induced cardiomyopathy” refers to dysfunction of cardiac muscle tissue in a subject following administration of an anthracycline, as defined herein, to the subject. In some embodiments, the dysfunction is chronic left ventricular dysfunction. Examples of signs of anthracycline-induced cardiomyopathy in a subject administered an anthracycline include, without limitation, sinus tachycardia, low QRS voltage, non-specific ST-T wave charges and/or poor R-wave progression across precordial leads (as determined by electrocardiogram); normal cardiac silhouette size with pleural effusions, venous encouragement and/or pulmonary edema (as determined by chest radiograph); and ejection fraction with normal left ventricular mass with septal dyskinesis, mitral regurgitation with a characteristic posteriorly-directed jet and/or decreased left ventricular compliance (as determined by echocardiography).

It is expected that during the life of a patent maturing from this application many relevant techniques for diagnosing an anthracycline-induced cardiomyopathy will be developed and the scope of the term “anthracycline-induced cardiomyopathy” is intended to include all such new technologies a priori.

In some embodiments of any of the embodiments described herein relating to anthracycline-induced cardiomyopathy, the cardiomyopathy is associated with fibrosis, for example, left ventricular fibrosis.

In some embodiments of any of the embodiments described herein relating to anthracycline-induced cardiomyopathy and/or fibrosis associated with an anthracycline, the subject is a subject who has received a cumulative dose of at least 450 mg/m² of an anthracycline, optionally doxorubicin.

In some embodiments of any of the embodiments described herein relating to anthracycline-induced cardiomyopathy and/or fibrosis associated with an anthracycline, the left ventricle exhibits dysfunction, yet is normal-sized, without dilatation.

Without being bound by any particular theory, it is believed that a normal-sized yet dysfunctional left ventricle is characteristic of anthracycline-induced cardiomyopathy, and that the absence of dilatation is indicative of fibrosis.

In some embodiments of any of the embodiments described herein, the compound or method according to any of the aspects of the embodiments described herein is for the treatment of a subject diagnosed with said fibrosis, e.g., a diagnosis of the fibrosis precedes the treatment.

In some embodiments of any of the embodiments described herein, the subject is treated with an anthracycline and is diagnosed with cardiomyopathy as described herein following the treatment with the anthracycline.

In some embodiments of any of the embodiments described herein relating to a method of treatment, the method further comprises administering an anticancer therapy (e.g., administering an anticancer drug) according to any of the respective embodiments described herein to the subject during a first time period; following the first time period, determining whether the subject is afflicted with fibrosis; and administering the compound of general Formula I (according to any of the respective embodiments described herein) to a subject determined to be afflicted with fibrosis during a second time period.

In some embodiments of any of the embodiments described herein relating to a method of treatment, the method further comprises administering an anthracycline according to any of the respective embodiments described herein to the subject during a first time period; following the first time period, determining whether the subject is afflicted with cardiomyopathy; and administering the compound of general Formula I (according to any of the respective embodiments described herein) to a subject determined to be afflicted with cardiomyopathy during a second time period.

In some embodiments of any of the embodiments described herein, the compound or method described herein is for prophylactic use in combination with an anticancer therapy (e.g., administration of an anticancer drug) according to any of the respective embodiments described herein, e.g., the treatment is for reducing a risk of development of an inflammatory disease or disorder (e.g., fibrosis) in a subject receiving the anticancer therapy.

In some embodiments of any of the embodiments described herein, the subject is treated with an anthracycline as described herein which is co-administered with the compound of general Formula I (e.g., prophylactic use of the compound).

In some embodiments of any of the embodiments described herein relating to a method of treatment, the method further comprises administering the anticancer therapy (e.g., anticancer drug) according to any of the respective embodiments described herein to the subject, and co-administering compound of general Formula I (according to any of the respective embodiments described herein) with the anticancer therapy. In some such embodiments, the method comprises co-administering an anthracycline with the compound of general Formula I.

Without being bound by any particular theory, it is believed that the therapeutic properties exhibited by the exemplary compound AN446 (as described herein) are at least in part associated with HDAC inhibition exhibited by AN446 and/or by valproic acid released by hydrolysis of AN446. It is further believed that AN446 exhibits superior anti-inflammatory (e.g., anti-fibrosis) properties, superior anticancer properties, superior bioavailability and/or lower toxicity, as compared to alternative HDAC inhibitors such as trichostatin A, entinostat and Tubastatin A (all of which have unacceptable toxicity), as well as curcumin, polyphenolic compounds, butyric acid, valproic acid and phenylbutyric acid (which have little or no anticancer potency).

In some embodiments of any of the embodiments described herein, X in Formula I is O (an oxygen atom).

In some embodiments of any of the embodiments described herein, R₂ is hydrogen.

In some embodiments of any of the embodiments described herein, R₁ is H or —CH₂OR₃.

In some embodiments of any of the embodiments described herein, R₁ is hydrogen (H). In some such embodiments, X is O. Compounds in which R₁ is hydrogen and X is O may be considered as derivatives of acyclovir.

In some embodiments of any of the embodiments described herein, R₁ is —CH₂OR₃. In some such embodiments, X is O. In some such embodiments, X is CH₂. Compounds in which R₁ is —CH₂OR₃ and X is O may be considered as derivatives of ganciclovir. Compounds in which R₁ is —CH₂OR₃ and X is CH₂ may be considered as derivatives of penciclovir.

In some embodiments of any of the embodiments described herein, the compound is N-valproyl-9-(2-hydroxy)ethoxymethyl-guanine, such that X is an oxygen atom, and R₁ and R₂ are each hydrogen.

The compound N-valproyl-9-(2-hydroxy)ethoxymethyl-guanine is referred to herein interchangeably as “AN446” and “valprostat”.

The present embodiments further encompass any pharmaceutically acceptable salts, hydrates and solvates of the compounds described hereinabove.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound. An example, without limitation, of a pharmaceutically acceptable salt would be an ammonium anion and an anion such as, but not limited to, chloride.

The term “solvate” refers to a complex of variable stoichiometry (e.g., di-, tri-, tetra-, penta-, hexa-, and so on), which is formed by a solute (the compound of present embodiments) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

As is detailed herein, the newly designed compounds of present embodiments exert highly beneficial anti-inflammatory and/or cardioprotective activities and therefore can be utilized in various therapeutic applications. Utilizing these compounds in therapeutic application involves administration thereof either per se, or as a part of a pharmaceutical composition where it is mixed with suitable carriers or excipients.

In some embodiments of any one of the embodiments described herein, there is provided a pharmaceutical composition, which comprises, as an active ingredient, a compound having general Formula I according to any of the respective embodiments described herein, and a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compounds accountable for the biological effect of the composition.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

In an optional embodiment of the present invention, the pharmaceutical compositions are designed for modulating an immune and/or inflammatory response via mucosal administration.

In another optional embodiment of the present invention, the pharmaceutical compositions are designed for modulating an immune and/or inflammatory response via oral administration.

Optionally, the pharmaceutical compositions of embodiments of the present invention are designed for nasal or intraperitoneal administration, as is detailed herein.

Pharmaceutical compositions of embodiments of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with present embodiments thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, for example, in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to embodiments of the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethylcellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of embodiments of the present invention may also be formulated in rectal compositions such as suppositories or retention enemas, using, e.g., conventional suppository bases such as cocoa butter or other glycerides.

Pharmaceutical compositions suitable for use in context of the present embodiments include compositions wherein the active ingredients are contained in an amount effective to achieve the intended purpose. More specifically, a therapeutically effective amount means an amount of active ingredients effective to prevent, alleviate or ameliorate symptoms of a disorder (e.g., fibrosis and/or cardiomyopathy) or prolong the survival of the subject being treated.

Determination of a therapeutically effective amount is well within the capability of those skilled in the art, especially in light of the detailed disclosure provided herein.

For any preparation used in the methods of embodiments of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures (e.g., as exemplified herein in the Examples section) or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See, e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress an inflammatory process (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of embodiments of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation described herein formulated in a compatible pharmaceutical carrier may also be prepared, placed in an appropriate container, and labeled for treatment of an indicated condition, as is further detailed herein.

Thus, in an optional embodiment of the present invention, the pharmaceutical composition is packaged in a packaging material and identified in print, on or in the packaging material, for use in the treatment or prevention of an inflammatory disease or disorder, according to any of the respective embodiments described herein.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

AN446 (N-valproyl-9-(2-valproyloxy)ethoxymethyl-guanine) and AN7 (butyroxymethyl diethylphosphate) were synthesized according to procedures described in International Patent Application Publication WO 2008/120205 and Nudelman et al. [Eur J Med Chem 2001, 36:63-74]. The expected hydrolysis products of AN446 are valproic acid and N-valproyl-9-(2-hydroxy)ethoxymethyl-guanine, and the expected hydrolysis products of AN7 are butyric acid, formaldehyde, phosphoric acid and ethanol.

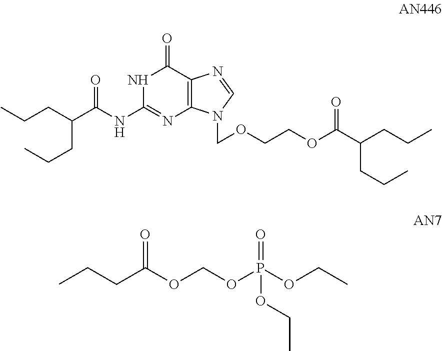

Doxorubicin hydrochloride (2 mg/ml) was obtained from Ebewe Pharma GmbH.

Rabbit polyclonal antibodies for c-Myc were obtained from Cell Signaling.

Rabbit polyclonal antibodies for p-c-Myc (Thr 58) were obtained from Santa Cruz Biotechnology.

Rabbit polyclonal antibodies for low molecular weight fibroblast growth factor (lo-FGF) were obtained from Santa Cruz Biotechnology.

Rabbit polyclonal antibodies for heme oxygenase (HO-1) were obtained from Stressgen.

Rabbit polyclonal antibodies for Ki67 were obtained from Lab Vision.

Rabbit polyclonal antibodies for actin were obtained from MP Biomedicals.

Mouse monoclonal antibodies against human VEGF were obtained from Santa Cruz Biotechnology.

Goat anti-mouse and anti-rabbit IgG antibodies (IRDye® 1680-labeled) were obtained from LI-COR Biosciences.

Biotinylated goat anti-mouse and anti-rabbit IgG-B antibodies were obtained from Santa Cruz Biotechnology.

Cell Cultures:

The murine mammary carcinoma 4T1, the embryonic rat heart H9C2, the human prostate carcinoma 22Rv-1, breast carcinoma MCF-7, MCF-7DX, T47D, myelocytic leukemia HL-60, T-cell lymphoma Jurkat, EBV(+) B-cell lymphoma Daudi and gastric carcinoma NCI-N87 were obtained from ATCC. The U251 MG human glioma cell line was obtained from ABGENT. Astrocyte cultures were prepared from brains of the same neonatal rats according to procedures described in Tarasenko et al. [Invest New Drugs 2012, 30:130-143]. All the cell lines were grown in DMEM, except for HL-60, Daudi and Jurkat that were grown in RPMI. The growth medium was supplemented with 10% fetal calf serum (FCS), 2 mM/1 glutamine, 100 units/ml penicillin, 100 mg/ml streptomycin, 12.5 units/ml nystatin (Biological Industries, Israel), and incubated in a humidified atmosphere of 5% CO₂ and 95% air at 37° C.

Viability Assays:

Cell viability by flow cytometry analysis (FACS) analysis was determined using the Mebcyto apoptosis kit (MBL-Japan). The cell lines 4T1, U251 (2×10⁵ cells/well), astrocytes and the cardiomyoblastic cell line H9C2 (5×10⁵ cells/well) were seeded in six-well plates. Following treatment they were trypsinized, washed with PBS, resuspended in 200 ml of binding buffer and double-stained with annexin V-FITC and propidium iodide (PI) according to the manufacturer's instructions. The stained cells were subjected to FACS analyses (Calibur cytometer, Becton Dickinson) where 30,000 cells were allowed to flow for each determination point and the percentage of cells in each state (viability or death) was defined by their distribution in fluorescence dot-plots using WinMdi 2.8 software (Scripps Research Institute, La-Jolla, USA).

Hoechst assay: 22RV-1, MCF-7, MCF-7DX, 4T1, U251, and T47D cells (3×10³ cells/well) and NCI-N87 (8×10³ cells/well) were seeded in tissue culture in growth medium for 24 h in 96-well plates (in triplicate) and then exposed to different concentrations (titration) of the tested drug for 72 h at 37° C. The plates were then rinsed with PBS and fixed with 100 ml of 70% ethanol for 30 min. The ethanol was decanted; 200 ml DNA binding dye Hoechst reagent, solubilized in DDW (10 mg/ml), was added. The fluorescence was measured (excitation at 390 nm and emission at 460 nm) with a Synergy III Fluorometer-Elisa reader (BioTek Instruments, Inc. Germany). The viability of cells grown in suspension was measured by the MTT assay. HL-60, Daudi and Jurkat cells were seeded (5×10³ cells/well) in 96-well plates in growth medium and immediately exposed to different concentrations (titration) of the tested drugs (in triplicate). After 72 h incubation, the MTT reagent in PBS (10 ml of 5 mg/ml) was added to the cell suspension and incubated for an additional 3 h. Then, 100 ml of 0.04 M HCl in isopropanol was added and the absorbance at 570 nm measured using a Synergy III Fluorometer-spectrophotometer ELISA reader.

Measurement of the Activity of HDAC:

U251 or Daudi cells seeded in 96-well plates (10⁴ cells/well, in quadruplicates) in growth medium for 24 h were then treated as indicated for 2 h in the presence of the HDAC substrate (Fluor de Lys™). The reaction was terminated by the addition of the Fluor de Lys™ developer (Biomol Research Labs) and 2 mM trichostatin A (TSA). All of the above reagents were included in a kit for measuring activities of HDACs of classes I and II (AK-503, Biomol Research Labs). The percentage inhibition was calculated from the ratio of the fluorescence (measured at 355 nm excitation and at 460 nm emission) in the drug treated compared with the untreated control culture.

Reactive Oxygen Species (ROS):

ROS were measured in live cells by monitoring the oxidation of DCF-DA (a cell-permeable probe), which undergoes deacetylation by cellular esterases and fluoresces upon oxidation by ROS. U251 MG (2×10⁵ cells/six-well plates), astrocytes or H9C2 (5×10⁵ cells/six-well plates) were treated as specified, incubated with 10 mM DCF-DA for 30 min at 37° C., washed with PBS and analyzed (10⁴ cells) by flow cytometry using a 488 nm excitation beam (Calibur cytometer, Becton Dickinson). The percentage of cells stained positively for DCF was determined with CellQuest software (BD Biosciences, San Jose, Calif., USA).

In Vivo Cancer Models:

The animal experiments were conducted according to the NCI Laboratory Animal Care Guidelines with the approval of the Tel Aviv University Committee for Animal Experimentation and the Israel Ministry of Health.

Xenograft of glioblastoma: 8- to 10-week-old male Hsd athymic FOXN (Harlan, Jerusalem, Israel) were inoculated sc in the flanks with 5×10⁶ U251 cells. Tumor length (L) and width (W) were measured with a caliper twice weekly. Tumor volumes were calculated according to the formula: (L×W2)/2. When the tumor was established (volume of 50-100 mm³) the mice were randomly divided into six groups treated with: vehicle control (saline); ip Dox 4 mg/kg once weekly; po AN446 25 mg/kg thrice a week; po AN7 50 mg/kg thrice a week; combination of AN446+Dox (4 mg/kg ip Dox once a week and 25 mg/kg po AN446 thrice a week); AN7+Dox (4 mg/kg ip Dox once a week and 50 mg/kg po AN7 thrice weekly). Untreated, non-bearing tumor naive mice were maintained under the same conditions. The experiment was terminated after 25 treatment days, the mice were sacrificed under CO₂ and their tumors and hearts were removed, weighed and divided into parts dedicated for immunohistochemistry (IHC) and the rest were frozen (−80° C.) for Western blot and Quantitative PCR (qRT-PCR) analyses.

Isolation of RNA and Protein:

Tumors and hearts of the mice were rapidly removed, frozen on dry ice and kept at −70° C. Tissue was then homogenized and RNA and proteins extracted using the NucleoSpin® RNA/Protein kit according to the manufacturer's protocol (Macherey-Nagel, GmbH & Co KG).

Quantitative PCR Amplification:

Total RNA (1 mg) was primed by oligo dT and reverse-transcribed by Verso cDNA kit according to the manufacturer's protocol (Thermo Scientific). Analyses of qPCR were performed with Kapa Sybr® Fast (Kapa Biosystems) on a Step One Plus thermocycler (Applied Biosystems). The comparative threshold method was used to calculate the relative gene expression. Values were normalized against glyceraldehyde 3-phosphate dehydrogenase (GAPDH). Expression of the GAPDH mRNA in each individual sample was used to normalize the dataset. The sequences of the primers were as follows:

| c-Myc (mouse): | |
|---|---|
| Forward | 50-CCAAATCCTGTACCTCGTC; |
| Reverse | 50-CCACAGACACCACATCAA |
| HO-1 (mouse): | |
| Forward | 50-TGAATCGAGCAGAACCAG |
| Reverse | 50-ATTCTCGGCTTGGATGTGTA |
| bFGF (mouse): | |
| Forward | 50-AAGGAAGATGGACGGCTG |
| Reverse | 50-CCAACTGGAGTATTTCCGTG |
| Collagen T1 (mouse): | |
| Forward | 50-ACAGCACCCTTGTGGAC |
| Reverse | 50-GCCAATGTCTAGTCCGAAT |
| GAPDH (mouse): | |
| Forward | 50-AGTCCATGCCATCACTGC |
| Reverse | 50-ACCTTGCCCACAGCCTT |
| HO-1 (human): | |
| Forward | 50-GGCAGAGAATGCTGAGTT |
| Reverse | 50-CTGCATGGCTGGTGTGTA |
| bFGF (human): | |
| Forward | 50-TATGAAGGAAGATGGAAGATTACTG |
| Reverse | 50-ATGTGAAATGAGATTAGATGTGG |
| Collagen T1 (human): | |
| Forward | 50-CAGCGTCACTGTCGATG |
| Reverse | 50-CCAACGTCGAAGCCG |
| GAPDH (human): | |
| Forward | 50-CTTTGGTATCGTGGAAGGACTC |
| Reverse | 50-AGTAGAGGCAGGGATGATGTTC |

The relative quantification of gene expression was determined by the comparative threshold method analyzing the relative gene expression data using real-time qPCR and the 2(-Delta Delta C(T)) Method, as described in Livak & Schmittgen [Methods 2001, 25:402-408].

Western Blot Analysis:

Protein levels in the samples were determined with the BCA protein assay kit (Pierce). The samples were subjected to Western blot analyses. The expression of proteins was visualized using their specified primary antibodies followed by the secondary IgG IRDye® 680DX antibody. Each detected band was quantified using the Odyssey Infrared Imaging System (LI-COR Biosciences) and normalized to the level of actin. The fold increase of a specific protein was determined by the ratio of the band intensity obtained from treated and untreated samples.

Immunohistochemistry:

The harvested organs were fixed in 4% paraformaldehyde for 24 h, washed with PBS, dehydrated in increasing alcohol concentrations and embedded in paraffin blocks, were processed according to procedures described in Tarasenko et al. [PLoS ONE 2012, 7:e31393] and Rephaeli et al. [Int J Cancer 2005, 116:226-235]. Inactivation of avidin-biotin nonspecific binding was prevented by a blocking kit, according to the manufacturer's protocol (Vector Laboratories). The sections were further incubated at 4 8° C. overnight with Ki-67 and VEGF antibodies. The secondary antibody was biotin conjugated goat anti-rabbit or anti-mouse IgG. Slides were then stained with the ABC peroxidase system, developed with diaminobenzidine (DAB) (Vector Laboratories) and counterstained with hematoxylin (Bio-Optica, Milano, Italy). The slides were examined using an Olympus BX 52 light microscope, and images were taken with an Olympus DP50 digital camera system.

Data Analysis:

A two-sided t-test between groups was performed using the Excel package for Windows 2007 (Microsoft). Survival was estimated by the Kaplan-Meier curves, and the difference in survival between the groups was analyzed by Wilcoxon's test and using a log-rank chi-square test. The Median Effect Analysis (MEA) for a constant combination ratio was used for drugs interaction and the combination index (CI) determination. The drug concentration dependence plots were generated for each of the drugs alone and in combination using CompuSyn software (ComboSyn, Inc.) developed by Chou et al., as described by Chou [Pharmacol Rev 2006, 58:621-681; and Cancer Res 2010, 70:440-446] and Engel et al., [J Cancer Res Clin Oncol 2006, 132:673-683].

### Example 1

### Activity of AN446 in Cancerous and Noncancerous Cells AN446 In Vitro Activities

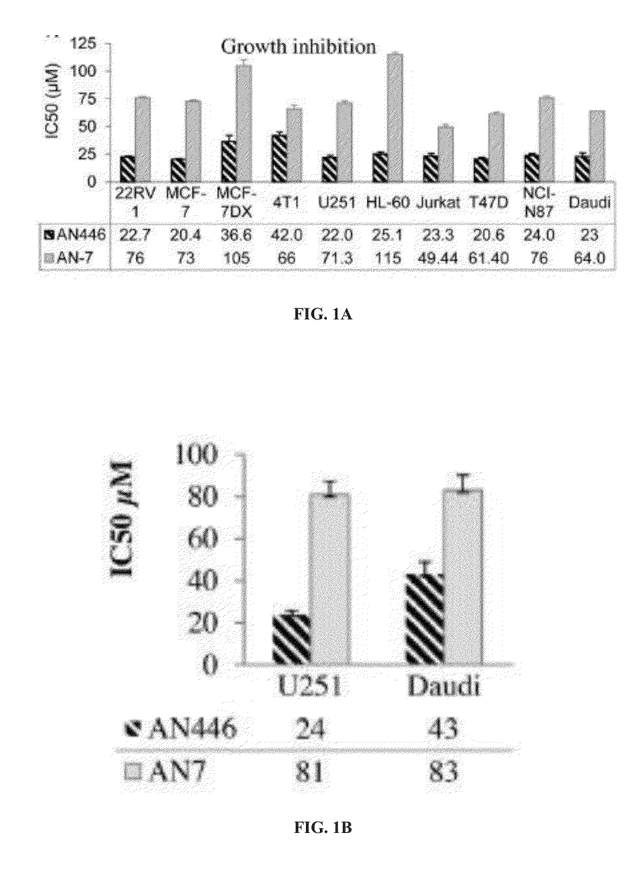

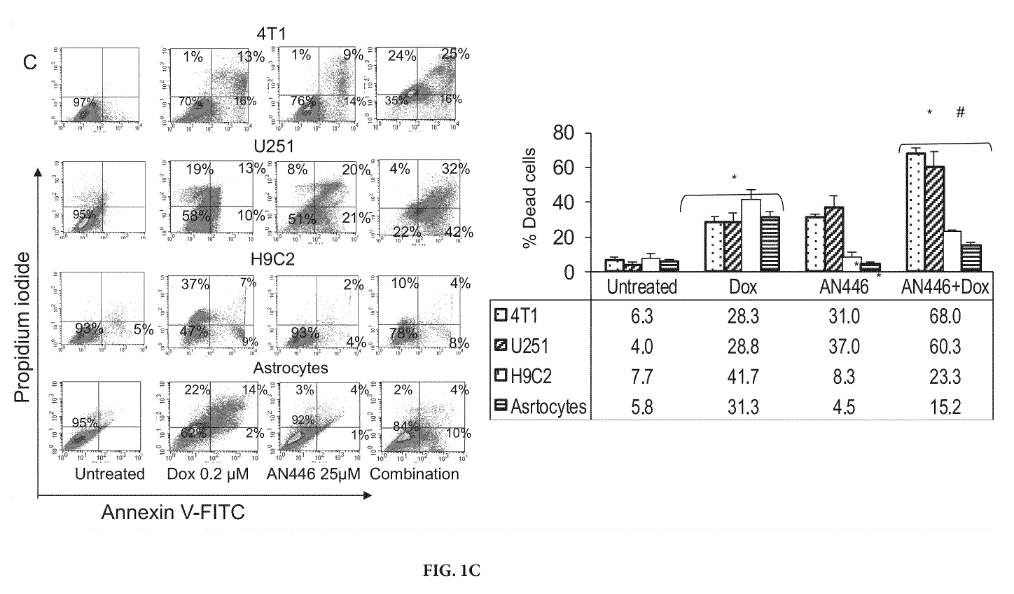

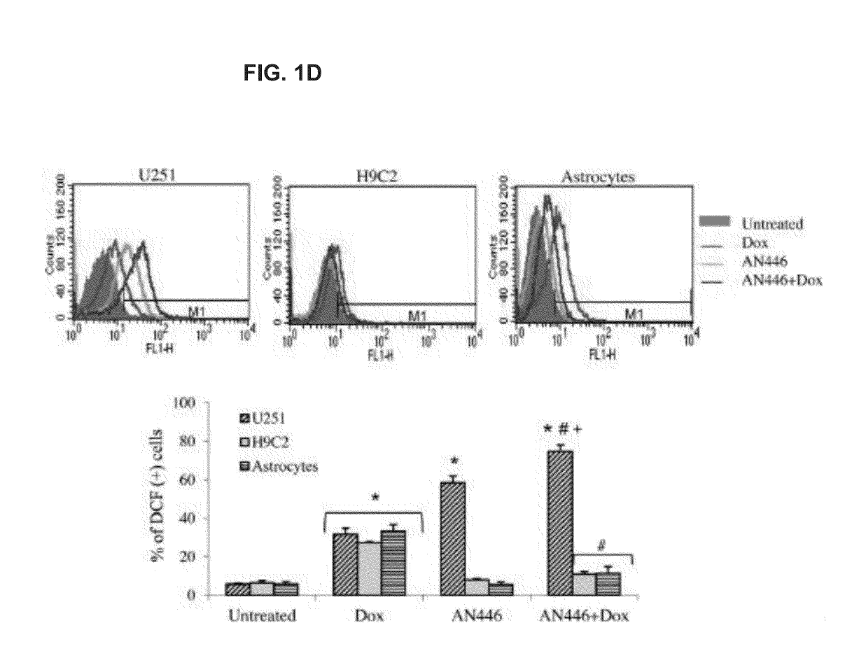

As shown in FIG. 1A, AN446 was a significantly more potent anticancer agent than AN7, as determined by a viability test performed with 10 different cancer cell lines. The IC50 values of AN446 were 20-46 mM, whereas those of AN7 were 50-105 mM, demonstrating that AN446 was significantly more potent than AN7 (˜2-5 fold) in reducing the viability of cancer cells.

As shown in FIG. 1B, AN446 was a 2-3-fold more potent HDAC inhibitor than AN7, in both U251 and Daudi cells.

The apoptotic effect of AN446, Dox and their combination was evaluated in U251 and 4T1 cancer cells, and in the non-cancerous rat cardiomyoblast cell line (H9C2) and in primary rat astrocytes, using procedures previously described for AN7 [Tarasenko et al., Invest New Drugs 2012, 30:130-143].

As shown in FIG. 1C, Dox treatment significantly reduced the viability of all cell types by increasing necrosis (as indicated by cells positive for PI and negative for annexin) and late apoptosis (as indicated by cells positive for PI and annexin), whereas treatment with AN446, as a single agent, significantly enhanced the mortality of U251 and 4T1 cells, but did not substantially affect the viability of H9C2 cells or astrocytes. As further shown therein, the combination of AN446 and Dox, compared to treatment with Dox alone, resulted in a response that was cell type-specific, i.e., the mortality of U251 and 4T1 cells increased significantly and that of the noncancerous cells decreased significantly.

To gain insights into the mechanism of cell-type specific effects, ROS production in cancer cells and in non-cancer cells after treatment for 5-6 h with AN446, Dox and AN446+Dox, were assessed by staining the cells with the membrane-permeable fluorescent dye DCF-DA.

As shown in FIG. 1D, AN446 increased the percentage of ROS positive U251 glioblastoma cells significantly more than Dox, and the combination AN446+Dox was significantly more toxic than either AN446 or Dox alone. As further shown therein, AN446 did not induce ROS production in the noncancerous H9C2 cells and the astrocytes, and moreover, AN446 attenuated ROS production and mortality in Dox-treated noncancerous cells H9C2 cells and the astrocytes, suggesting that AN446 protected the noncancerous cells against Dox-induced ROS-production and cell-damage.

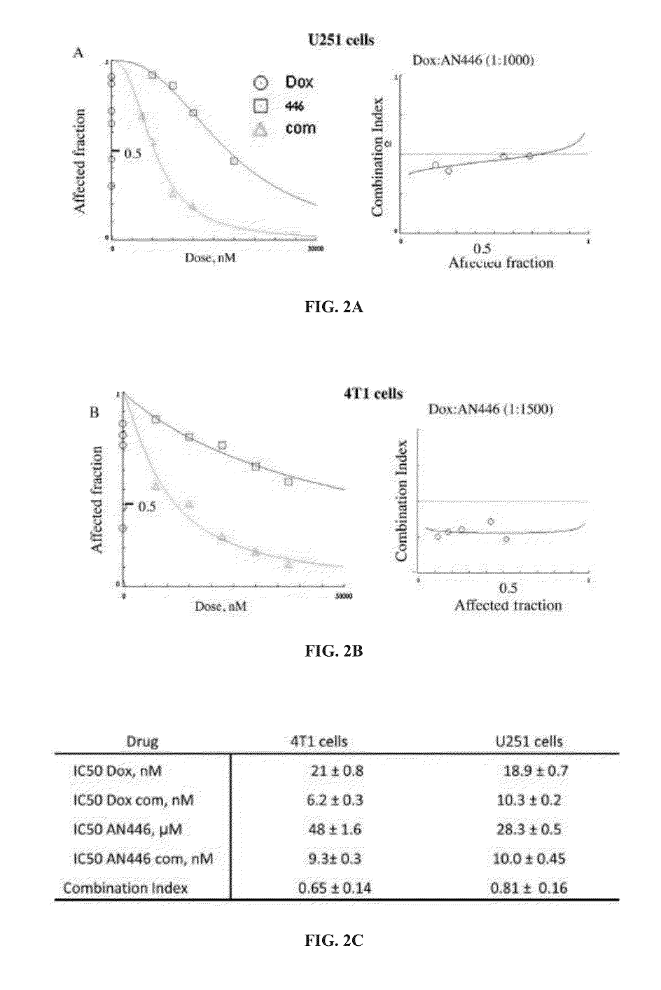

AN446 and Dox reduced the survival of the glioblastoma U251 and the breast carcinoma 4T1 cell lines, whereas AN446 in combination with Dox significantly increased cell mortality compared with single drug treatments. To determine the nature of AN446+Dox interactions and AN446+paclitaxel interactions, combination studies using a constant ratio of the drugs were conducted according to the MEA method, as described by Chou et al. [Pharmacol Rev 2006, 58:621-681]. The average values of three independent experiments were calculated and analyzed with CalcuSyn software. The combination of AN446 and Dox was tested in U251 and 4T1 cells at constant molar ratios of 1:1000 or 1:1500 (Dox/AN446), respectively. Dose-response-effect of the drugs on survival and combination index (CI) plots were generated by the software, and are presented in FIGS. 2A and 2B, and the combination indices (CIs) and the concentrations of the drugs as single agents and in combination (com) needed to achieve 50% survival are summarized in FIG. 2C.

As shown in FIGS. 2A-2C, in U251 cells the drugs interacted in synergy at high concentrations and additively at low concentrations, whereas in 4T1 cells, which were more resistant to AN446, the interaction was synergistic over a wide range of drugs concentrations.

Effect of the AN7, AN446 and their Combination with Dox on a Glioblastoma Xenograft Model:

Mice were implanted subcutaneously with U251 cells and when the tumors reached 50-100 mm³ treatments were initiated. Survival was evaluated by the Kaplan-Meier end-product analysis, where treatment failure (the experimental end points) was a loss of ˜20% body weight, or a tumor volume of >1 cm³. The experiment was terminated on day 25 of the treatment with the drugs, when 67% of mice in the vehicle-treated group (8 mice from a group of 12) failed treatment.

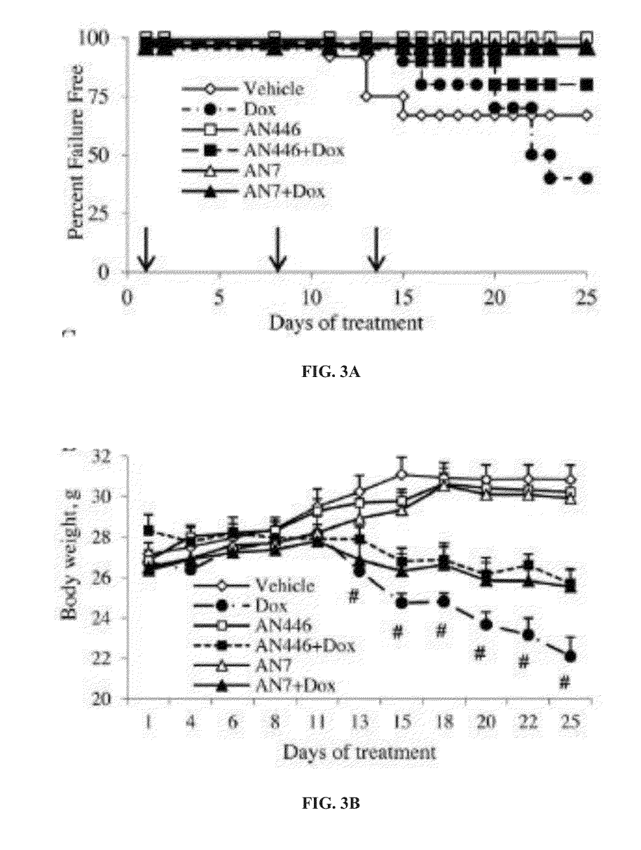

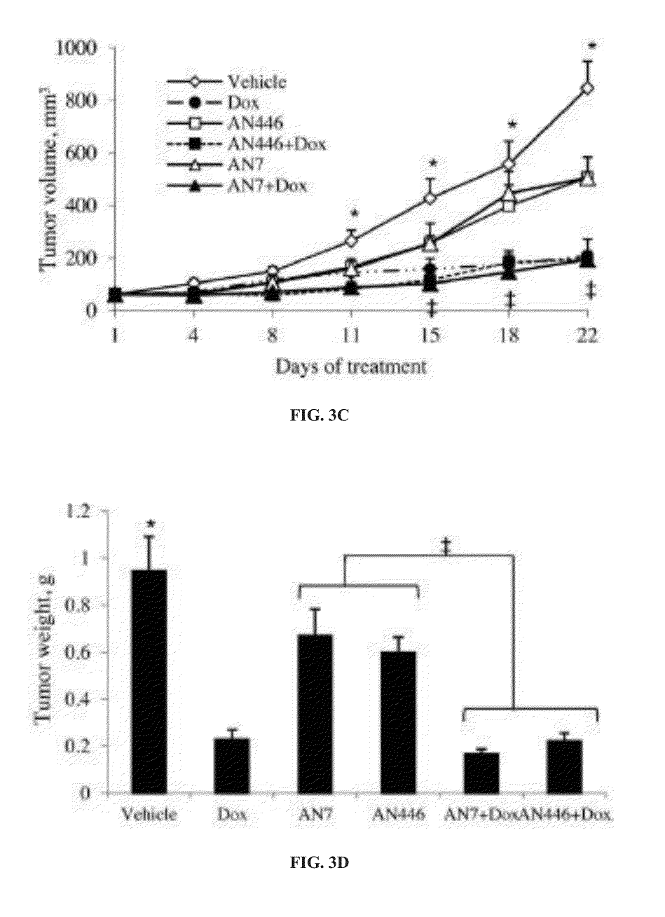

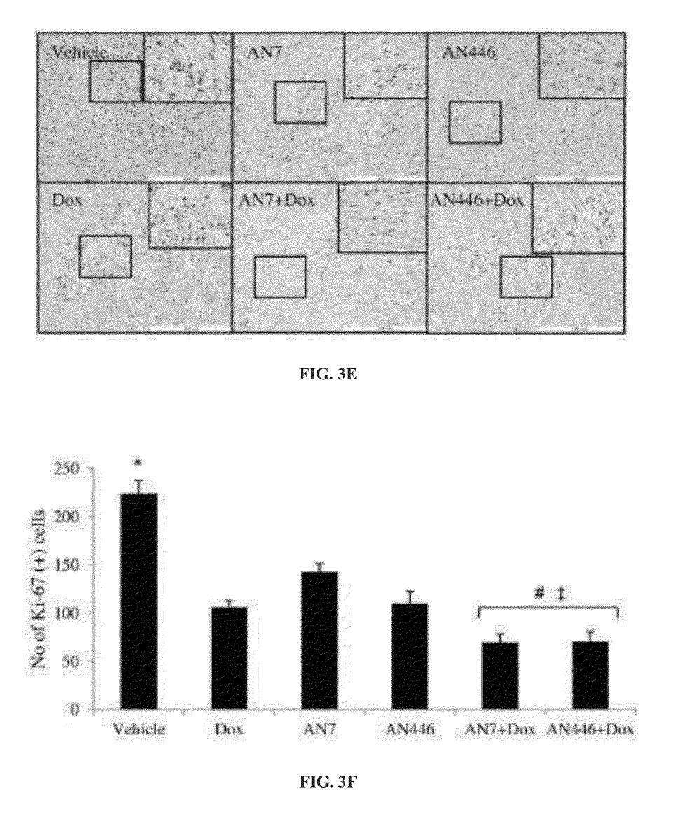

As shown in FIG. 3A, 100% (10/10) of the mice that received AN446 (25 mg/kg) or AN7 (50 mg/kg) thrice weekly survived, whereas only 40% (4/10) of those treated with Dox (4 mg/kg) once a week lived until the end of the experiment. As further shown therein, addition of AN446 (25 mg/kg thrice a week) to the Dox (4 mg/kg) treatment significantly prolonged (p<0.01) the survival of the mice to 80% (8/10), and addition of AN7 to Dox treatment resulted in 100% (10/10) survival of the mice.

Accompanying the high mortality in the Dox-treated group was a drastic decrease in body weight, seen from day 13 of the treatment. This weight loss, contributing to treatment failure in 60% of these mice, indicated toxicity.

As shown in FIG. 3B, a significant attenuation of body weight loss was achieved by the addition of AN446 or AN7 to the Dox treatment.

As shown in FIG. 3C, the tumor volume, from day 11 (mean±SE), was significantly higher in the vehicle-treated group compared with all other treatment groups, being 2-fold higher than of mice treated with AN446 or AN7 and 3-fold higher than those treated with AN446 or AN7 in combination with Dox.

On treatment failure, the tumors were harvested individually and weighed.

As shown in FIG. 3D, mean tumor weight was significantly higher in the vehicle-treated group compared with all other groups (p<0.05), and the tumor weight increase was significantly lower in the combination-treated groups compared to that of the AN446 or AN7 treated groups (p<0.05).

The nuclear antigen Ki-67, preferentially expressed in the proliferating cells, is a marker for the clinical evaluation of tumor proliferation.

As shown in FIGS. 3E and 3F, considerably more Ki-67 positive cells were detected in tumors of the vehicle-treated mice, than in tumors of the AN446-, AN7- or Dox-treated mice. As further shown in FIG. 3F, the combination of AN446 or AN7 with Dox reduced the proliferating cells to a level significantly lower than any of the single drug treatments.

These results indicate that AN7 and AN446 possess selective anticancer activity, augment Dox anticancer activity and ameliorate its toxicity in the described glioblastoma xenograft.

The effect of the drugs on the expression of genes associated with cell survival and death was examined by following changes in transcription and protein expression in tissue samples taken from the mice with glioblastoma xenografts. Heme oxygenase (HO-1) is known to play a protective role in the heart, whereas, in many cancers it is expressed and promotes growth and survival of neoplastic cells.

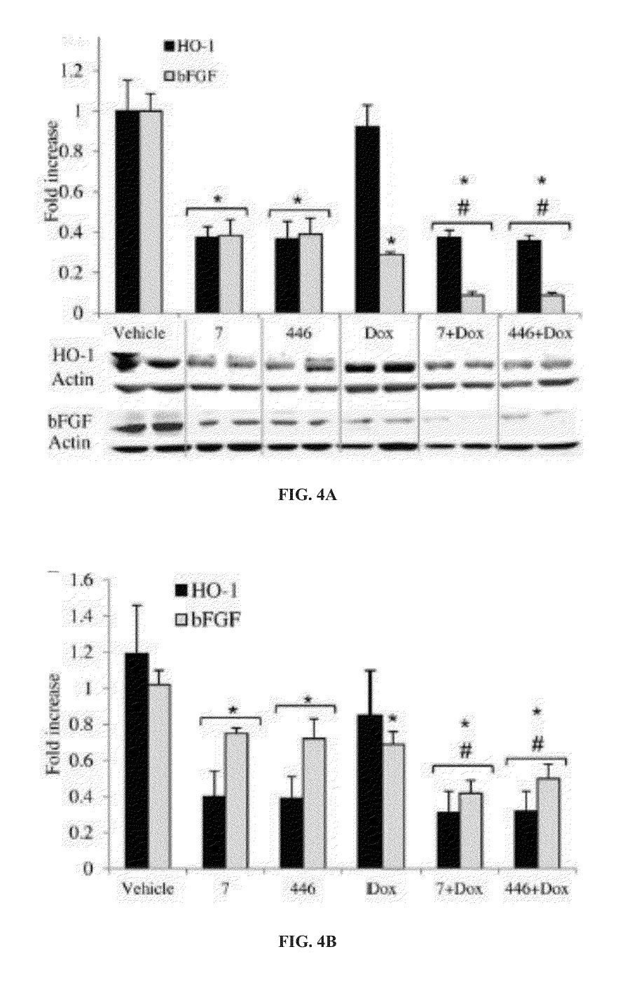

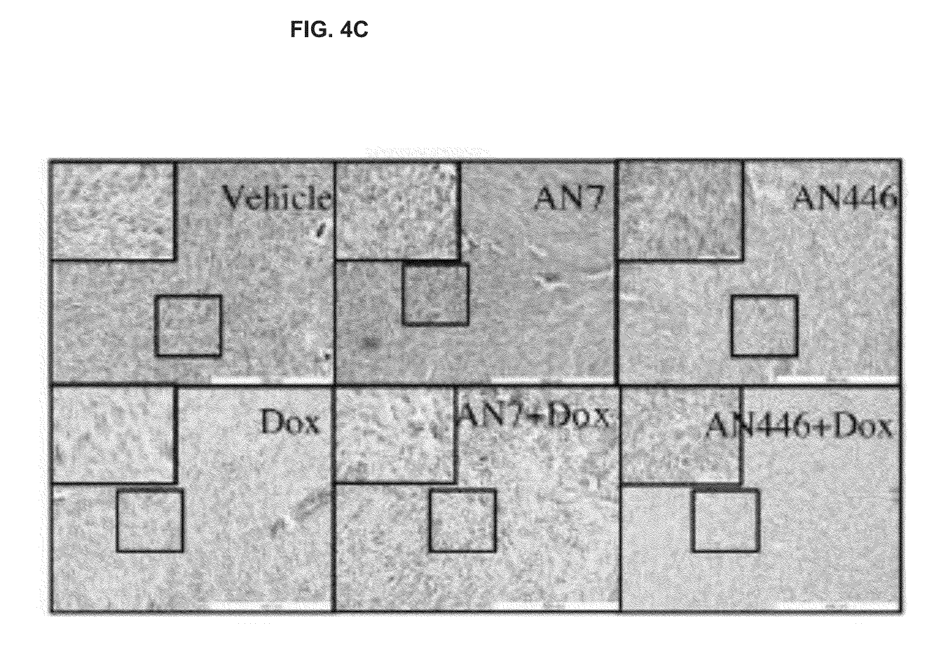

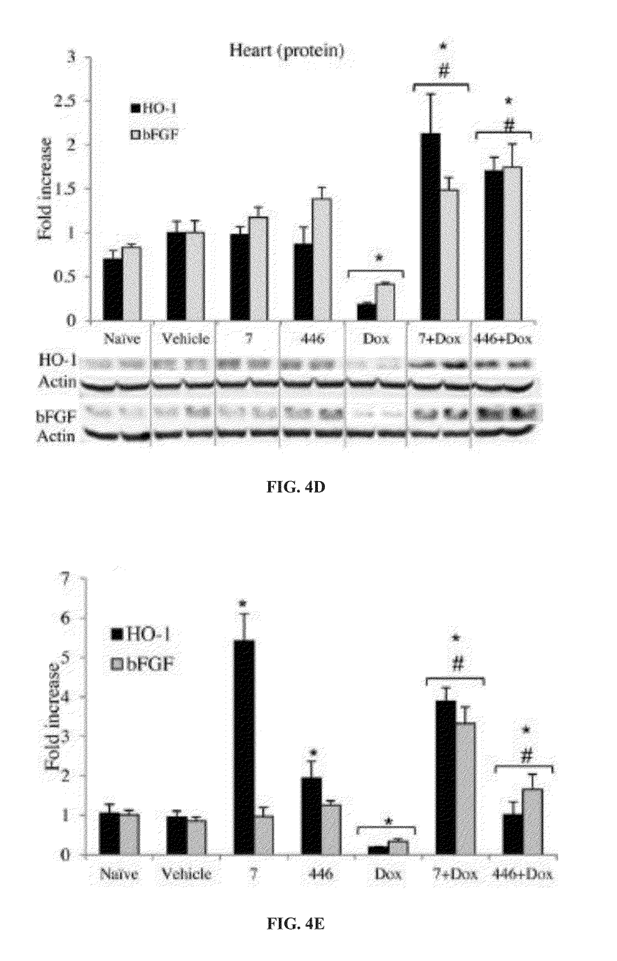

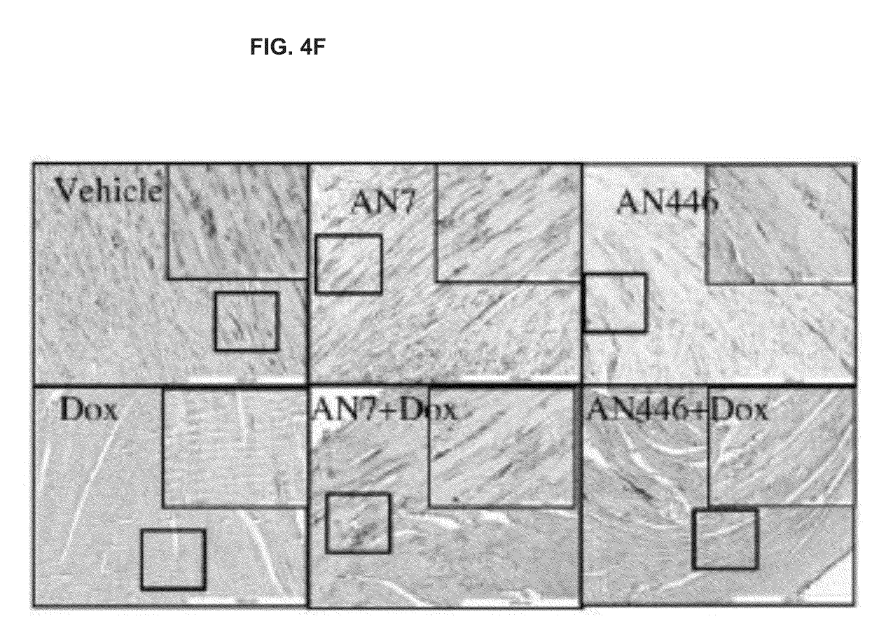

As shown in FIGS. 4A and 4B, tumor HO-1 protein expression was unchanged by Dox-treatment, but decreased by about 2-2.5-fold in response to treatment with AN446 or AN7, alone or in combination with Dox, and similar alterations were observed in the level of HO-1 transcript in the tumor.

These results indicate that in the combination of AN446 or AN7 with Dox, the inhibitory effect of the AN446 or AN7 prevailed.

As shown in FIGS. 4D and 4E, HO-1 expression in the heart did not significantly change upon treatment with AN446 or AN7, whereas Dox decreased it ˜5-fold. As further shown therein, co-treatment of Dox with AN446 or AN7 increased the expression of this enzyme to a significantly higher level than that observed in the hearts of naive or vehicle-treated mice. Similarly, Dox treatment decreased HO-1 mRNA, whereas co-treatment of Dox together with AN446 or AN7 significantly elevated its basal expression level by 3.89±0.35 fold and 1.76±0.36 fold, respectively.

The pro-angiogenic factors basic fibroblast growth factor (bFGF) and vascular endothelial growth factor (VEGF) are potent angiogenic stimulators. The expression of bFGF in the tumor and in the heart was evaluated by Western blot and qRT-PCR analyses and expression of VEGF by IHC.

As shown in FIGS. 4A and 4B, expression of bFGF in tumors was decreased significantly by AN446, AN7 and Dox, and further diminished by the combination of the AN446 or AN7 with Dox, thereby demonstrating the anti-angiogenic activity of AN446, AN7, Dox and combinations thereof. As further shown therein, changes in the level of bFGF transcript corresponded to those observed in protein levels.

As shown in FIG. 4C, VEGF expression in tumors was substantially reduced by all of the treatments.

In xenografts of glioblastoma it was examined whether the bFGF originated from the human glioblastoma tumor or the surrounding mouse microenvironment.

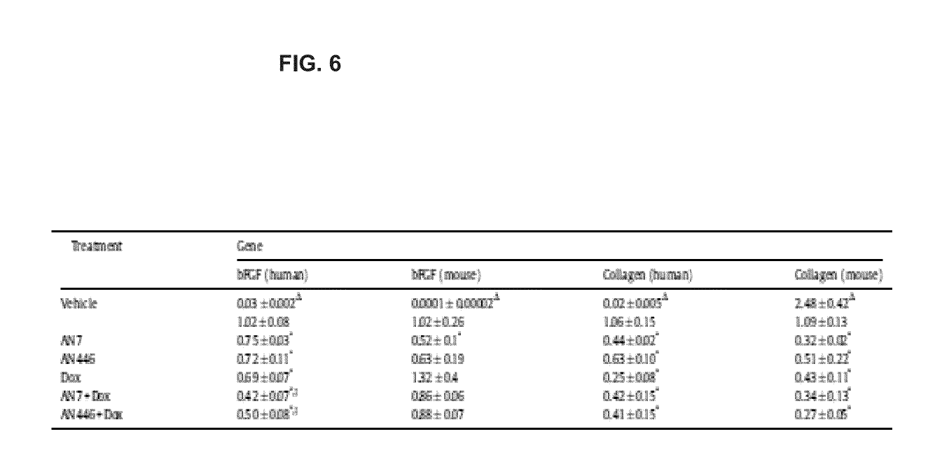

As shown in FIG. 6, the level of the human bFGF transcript was 30-fold higher than that of the mouse, indicating that the bFGF in the tumor was produced by the human glioblastoma cells and not by the mouse.

As further shown in FIG. 6, in the glioblastoma tumor, the presence of collagen T1 mRNA (which is typically produced by fibroblasts) from mouse was 100 fold higher than that of human, strongly supporting the notion that mouse fibroblasts populated the human glioblastoma tumor, yet the bFGF was produced by the glioblastoma cells.

As further shown in FIG. 6, treatment with the AN446, AN7 and Dox significantly reduced the bFGF mRNA in the tumor, and the combined treatment of AN446 or AN7 with Dox suppressed it significantly further. As further shown therein, collagen T1 mRNA level was significantly reduced by all drug treatments.

As shown in FIG. 4D, the expression of mouse bFGF protein was unchanged by AN446 or AN7 but decreased significantly by Dox treatment, whereas co-treatment of Dox with AN446 or AN7 significantly increased its expression to a level higher than that observed in naive or vehicle-treated mice (p<0.05).

As shown in FIG. 4E, the changes in the level of bFGF transcript in the heart corresponded to that of protein expression.

As shown in FIG. 4F, VEGF expression in the heart was reduced by Dox and (as was observed with bFGF) it was elevated by treatment with AN446 or AN7.

Taken together, these results indicate that the pro-survival HO-1 and the angiogenic factors bFGF and VEGF were reduced by the prodrugs and their combination with Dox, whereas in the hearts they were elevated.

The c-Myc protein is an important regulator of normal cell physiology and in tumor cells it plays an important role in activating oncogenic pathways [Soucek & Evan, Curr Opin Genet Dev 2010, 20:91-95].

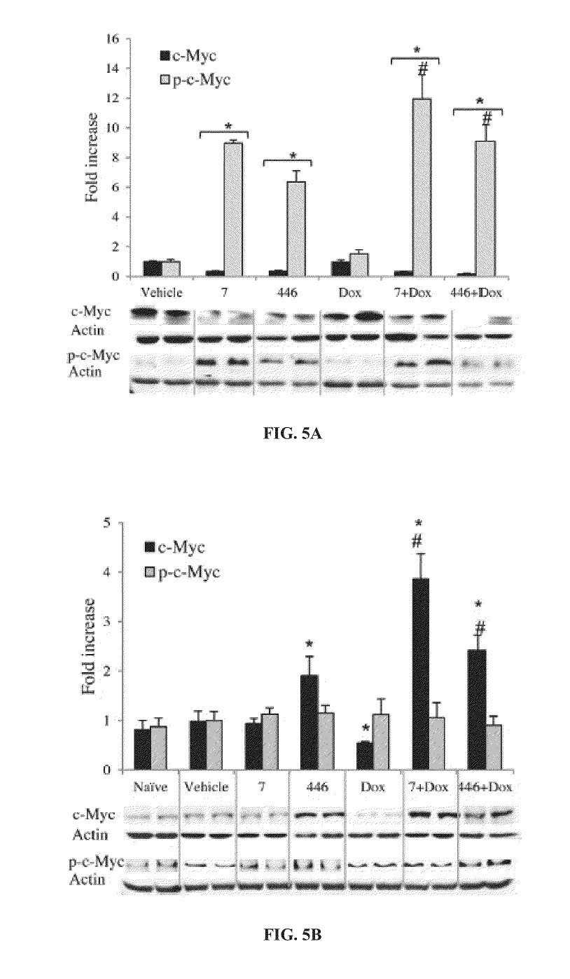

As shown in FIG. 5A, the high expression level of c-Myc protein in the tumors of vehicle-treated mice was unaffected by Dox, but decreased dramatically upon AN446, AN7, AN446+Dox and AN7+Dox treatment. The down-regulation was accompanied by a 7-12 fold increase in the level of c-Myc phosphorylated on threonine 58 (Thr58 p-c-Myc).

A significant body of evidence points to the importance of this phosphorylation in controlling the degradation of c-Myc [Vervoorts et al., J Biol Chem 2006, 281:34725-34729].

As shown in FIG. 5B, c-Myc protein expression in the hearts of the same mice was not affected by AN7, was repressed by Dox whereas AN446, AN446+Dox and AN7+Dox significantly increased its expression to a level higher than in naive or vehicle-treated mice. The low expression of p-c-Myc in the hearts of all mice was unchanged by all treatments.

These results indicate that AN446 and AN7, and their combination with Dox (but not Dox alone), reduced c-Myc levels in the tumor. In contrast Dox reduced c-Myc in the heart, whereas the combination of AN446 or AN7 with Dox significantly increased it.

### Example 2

### Effect of AN446 on Inflammation-Associated Pathways

The effect of AN446 on inflammation-associated pathways was evaluated in the monocytic THP1 cell line and in the MDA-MB-231 breast carcinoma cell line. Supernatants of treated THP1 cells were collected and IL-6, IL-10 and TNF-α concentrations were quantified by ELISA (R&D systems). TLR4 expression in THP1 cells was assessed using fluorescent anti-TLR4 antibodies and FACS analysis. In addition, gene expression for MCP-1, TNF-α and TLR4 in MDA-MB-231 was evaluated by qRT-PCT, following treatment with AN446 and/or paclitaxel.

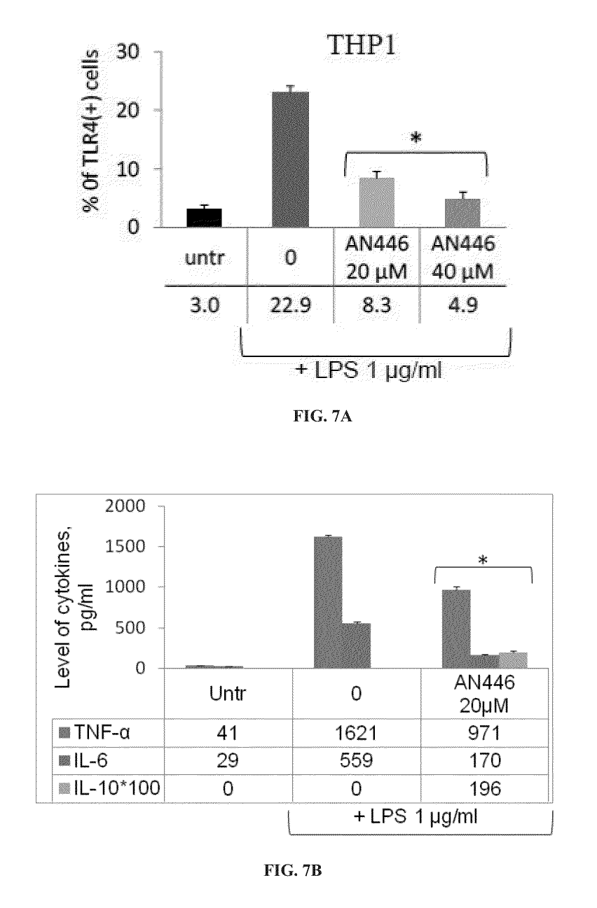

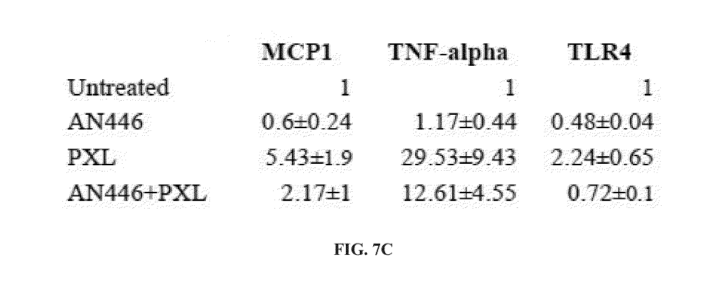

As shown in FIGS. 7A and 7B, AN446 suppressed the lipopolysaccharide (LPS)-induced expression of TLR4 (FIG. 7A) and the pro-inflammatory cytokines IL-6 and TNF-α (FIG. 7B) in THP1 cells. As further shown in FIG. 7B, AN446 also doubled the expression of the anti-inflammatory cytokine IL-10.

As shown in FIG. 7C, AN446 reduced expression of TLR4 in MDA-MB-231 cancer cells, which normally overexpress TLR4. As further shown therein, AN446 significantly attenuated the paclitaxel-induced increase in the expression of TLR4 and the pro-inflammatory factors MCP-1 and TNF-α.

These results indicate that AN446 has therapeutic potential in the treatment and prevention of various inflammatory conditions, including cancer development and inflammation-induced fibrosis.

### Example 3

### Effect of AN446 on Fibrosis and Inflammation-Associated Pathways in a Glioblastoma Xenograft Model

AN446 was tested in vivo using a xenograft of glioblastoma (U251 cells) in mice.

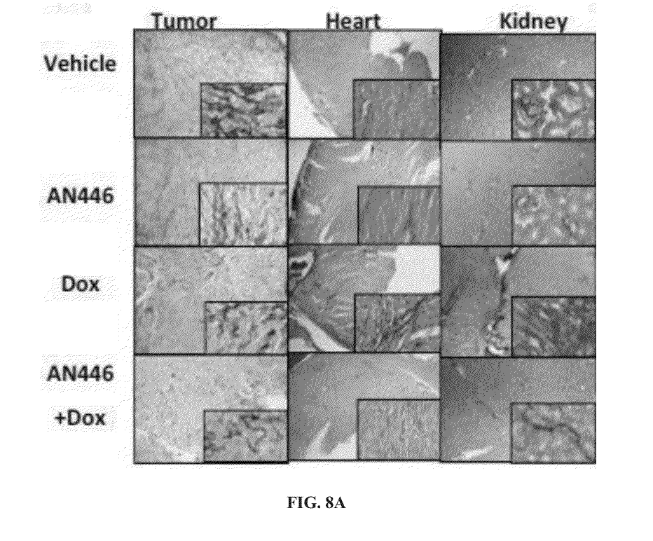

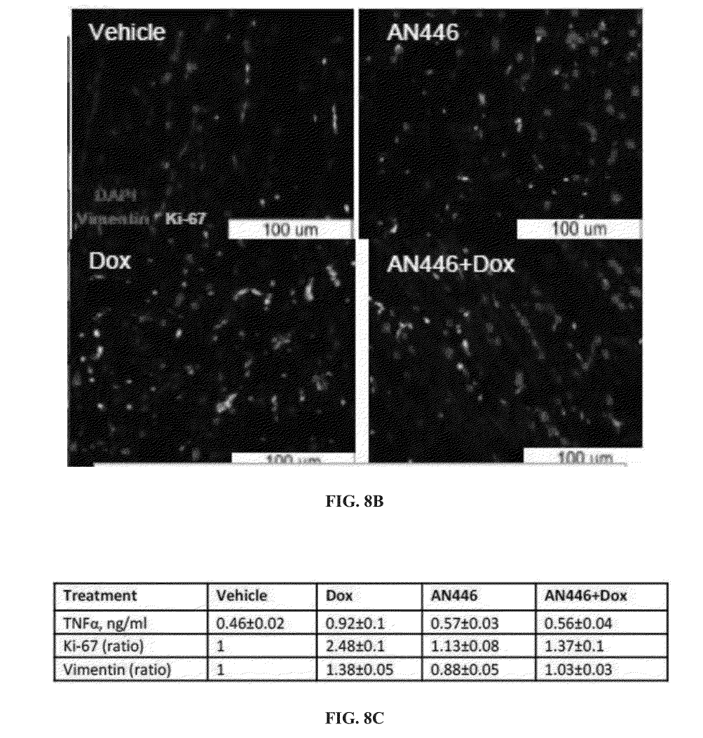

As shown in FIGS. 8A-8C, doxorubicin induced fibrosis (increased levels of fibroblasts) in the heart (FIGS. 8A-8C) and in the kidneys (FIG. 8A), and increased serum levels of TNF-α, a pro-inflammatory cytokine (FIG. 8C), whereas co-administration of AN446 with doxorubicin attenuated the doxorubicin-induced fibrosis in the heart (FIGS. 8A-8C) and the kidneys (FIG. 8A), and prevented a doxorubicin-induced rise in serum levels of TNF-α (FIG. 8C).

As further shown in FIG. 8A, untreated tumor was rich in fibroblasts, which harbor growth factor that can nourish the tumor, whereas the addition of doxorubicin, AN446 or their combination reduced fibrosis, thereby limiting the tumor growth. In addition, by double-staining heart sections by immunostaining for the fibroblast marker vimentin and the proliferation marker Ki67, it was observed that most of the proliferating (Ki67-positive) cells in hearts of doxorubicin-treated mice were fibroblasts (vimentin-positive).

Furthermore, as shown in FIGS. 8B and 8C, co-administration of AN446 with doxorubicin significantly reduced the doxorubicin-induced proliferation of fibroblasts in cardiac tissue.

These results indicate that AN446 ameliorates anthracycline-induced fibrosis and inflammation in tumors and in noncancerous tissue. Reduction of levels of fibroblasts in a tumor disrupts the tumor's supportive microenvironment, whereas concomitant reduction of levels of fibroblasts in the heart and kidney helps to preserve the normal function of these organs.

### Example 4

### Comparison of Effects of AN446 and Vorinostat (SAHA) on Cancerous and Noncancerous Cells

In order to evaluate the anticancer efficacy and selectivity of AN446, the effects of AN446 on cancerous and noncancerous cells were compared to those of vorinostat (suberanilohydroxamic acid, also known as “SAHA”), an HDAC inhibitor which is approved for treatment of cutaneous T-cell lymphoma (CTLC, a non-Hodgkin lymphoma of T lymphocytes residing in the skin), yet exhibits low response and significant adverse effects. The MyLa CTLC cell line (originating from an indolent mycosis fungoides patient) and the Hut-78 CTLC cell line (originating from an aggressive Sézary patient) were used to evaluate toxicity towards cancer cells, and peripheral blood lymphocytes (PBLs) from healthy volunteers were used to evaluate toxicity towards noncancerous cells.

Cell viability was determined by an MTT cell viability assay after 48 hours. MyLa cells (10⁴ cells/well), Hut78 cells (5×10³ cells/well) and normal PBLs (10⁵ cells/well) were seeded in 96 well-plates. The PBLs were activated with 40 μg/10⁶ cells of phytohemagglutinine (PHA) for 24 h before the experiment.

As shown in Table 1, AN446 was about 5-fold more toxic to the MyLa cells and about 10-fold more toxic to the Hut-78 cells than to normal PBLs, whereas SAHA was more toxic to PBLs than to MyLa cells, and only moderately more toxic to Hut-78 cells than to PBLs.

TABLE 1
IC₅₀ of SAHA and AN446 in MyLa and HUT78 CTCL cell
lines and in peripheral blood lymphocytes (PBL)

| | IC₅₀ (μM) | | |
|---|---|---|---|
| Compound | MyLa | HUT78 | PBL |
| SAHA | 4.3 ± 0.18 | 0.7 ± 0.05 | 2.5 ± 0.1 |
| AN446 | 40 ± 2.3 | 21 ± 1.3 | 200.3 ± 3 |

These results indicate that AN446 is more selective at killing cancer cells and minimizing toxicity towards noncancerous cells, as compared to SAHA (vorinostat).

As discussed hereinabove, AN446 synergistically augmented the anticancer efficacy of Dox while reducing Dox toxicity to noncancerous cells. The combination of 446 and Dox, compared to treatment with each drug alone, resulted in a response that was cell-type-specific. The effect of combination of AN446 with Dox was evaluated in several cancerous cell lines and in PBLs, by determining a combination index as described herein, and compared with the effect of combination of SAHA with Dox.

The interaction between AN446 or SAHA with Dox was evaluated in PBLs and cancer cells according to the median-effect method. The ratios of the drug combination were based on the ratio of their IC₅₀ of each drug alone. Cell viability was determined by MTT assay, as described hereinabove. The drugs were added to the cells and incubated for 72 h.

Drug interaction in adherent cells was evaluated as above except that the viability was determined by Hoechst assay. The cells MDA-MA-231 (3×10³ cells/well), T47D (3×10³ cells/well) and NCI-N87 (5×10³ cells/well) were seeded according to procedures known in the art.

As shown in Table 2, AN446 interacted synergistically with Dox (as indicated by CI values of less than 1) in each of the following human cancer cell lines: MDA-MB-231, T47D, NCI-N87 gastric carcinoma and HUT-78 CTCL cells, and antagonized the toxicity of Dox towards noncancerous PBLs (as indicated by a CI value of more than 1), whereas SAHA augmented the toxicity of Dox (CI>1) towards PBLs and towards 2 of 3 tested cell lines (MDA-MB-231 and HUT-78).

TABLE 2
IC₅₀ values and combination indices
(CI) for drug interactions with Dox

| | Cells | | | | |
|---|---|---|---|---|---|
| Drugs | MDA-231 | T47D | NCI-N87 | HUT-78 | PBL |
| SAHA IC₅₀ (μM) | 0.1 | 0.64 | ND | 0.64 | 2.50 |
| SAHA com IC₅₀ (μM) | 0.63 | 0.13 | ND | 0.63 | 0.50 |
| Dox IC₅₀ (nM) | 10.2 | 19.1 | ND | 31 | 70 |
| Dox com IC₅₀ (nM) | 4.3 | 5.2 | ND | 12 | 20 |
| CI (SAHA + Dox) | 1.1 | 0.5 | ND | 1.35 | 0.5 |
| AN446 IC₅₀ (μM) | 38 | 21.7 | 20 ± 3 | 28 | 198 |
| AN446 com IC₅₀ (μM) | 17 | 5.2 | 6 ± 1.5 | 11 | 187 |
| Dox IC₅₀ (nM) | 10.2 | 19 | 30 ± 1.3 | 30.8 | 70 |
| Dox com IC₅₀ (nM) | 5.3 | 7.6 | 8.5 ± 1.0 | 11 | 15 |
| CI (AN446 + Dox) | 0.8 | 0.7 | 0.6 | 0.8 | 1.2 |

These results are consistent with the results described above with respect to the effects of AN446 on noncancerous cardiac cells and astrocytes, and provide further indication that AN446 interacts with anticancer drugs in a strongly synergistic manner which is not characteristic of other HDAC inhibitors.

### Example 5

### Comparison of Effects of HDAC Inhibitors on Cancerous and Noncancerous Cells

The activity of valprostat (AN446) was compared to that of two FDA approved HDAC inhibitors, SAHA and romidepsin, and two clinically tested HDAC inhibitors, entinostat and valproic acid.

The effect of 24, 48 and 72 hour exposure to HDAC inhibitors on the viability of non-transformed cardiomyoblasts (H9C2 cells) and U251 human glioblastoma cells was tested using FACS analysis, and the results of three independent experiments were averaged. Each HDAC inhibitor was used at a concentration equal to its IC₅₀ value for viability in U251 cells after 72 hours, which was determined by evaluating viability according to the procedures described hereinabove.

As shown in FIG. 9 valprostat (AN446) was effective at inducing cell death in glioblastoma cells after 24, 48 or 72 hours, and was particularly more effective than the other tested HDAC inhibitors at inducing cell death after 24 hours, indicating that valprostat induced cell death more rapidly than did the other HDAC inhibitors.

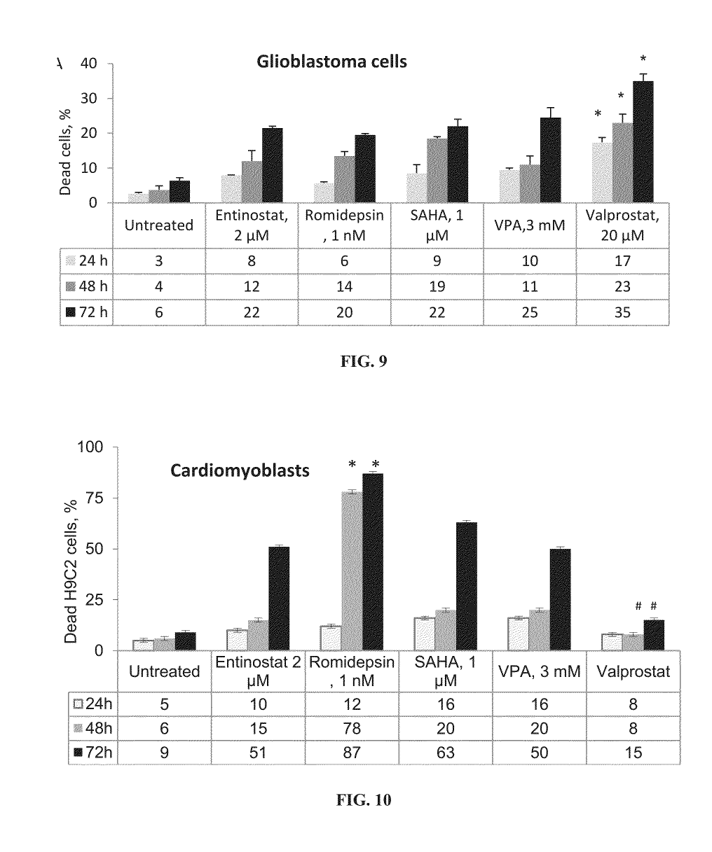

In contrast, as shown in FIG. 10, valprostat was less lethal than each of the other tested HDAC inhibitors in cardiomyocytes. Valprostat did not induce significant cardiomyocyte cell death after 24, 48 or 72 hours, whereas all other tested HDAC inhibitors induced significant cardiomyocyte cell death after 72 hours.

Therapeutic indexes (TI) of the HDAC inhibitors were calculated based on the ratio of cancer cell mortality to cardiomyoblast mortality after 24, 48 and 72 hours (as shown in FIGS. 9 and 10, respectively).

As shown in FIG. 11, valprostat exhibited a considerably higher therapeutic index than did the other HDAC inhibitors. The therapeutic index of valprostat was more than 2 at each time point, indicating consistently higher toxicity to the cancer cells than to the non-cancer cells; whereas all other tested HDAC inhibitors exhibited a therapeutic index of less than 1 at each time point, indicating consistently higher toxicity towards the non-cancer cells.

The effects of the abovementioned HDAC inhibitors on histone acetylation were also compared. U251 and H9C2 cells were treated with the HDAC inhibitors for 24 hours (according to procedures described hereinabove with respect to FIGS. 9 and 10), and then subjected to Western blot analysis.

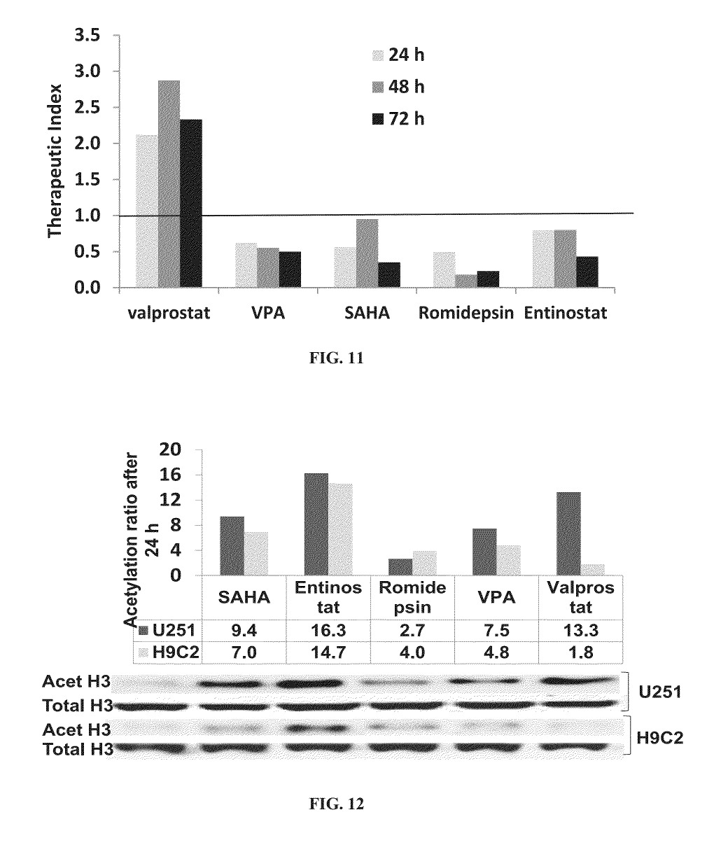

As shown in FIG. 12, valprostat (AN446) increased histone acetylation considerably in cancer cells but not in non-cancerous cardiomyocytes, whereas all other HDAC inhibitors increased histone acetylation in both cancer cells and cardiomyocytes to similar degrees.

These results indicate that the HDAC inhibitory activity of AN446 is selective towards cancer cells, which may advantageously help reduce deleterious cytotoxicity toward non-cancerous cells in cancer treatment.

The effects of the abovementioned HDAC inhibitors on DNA damage and repair were then assessed by evaluating changes in levels of phosphorylated histone H2AX (pH2AX), a marker of DNA double-strand breaks, and Rad51, a marker of homologous recombination-guided DNA repair of double-strand breaks [Nagathihalli & Nagaraju, Biochim Biophys Acta 2011, 1816:209-218; Rogakou et al., J Biol Chem 1998, 273:5858-5868].

U251 and H9C2 cells were treated with the HDAC inhibitors for 24 or 48 hours, and then subjected to Western blot analysis. Expression of pH2AX and Rad51 was detected with a specific antibody and quantified according to procedures described in Tarasenko et al. [PLoS ONE 2012, 7:e31393].

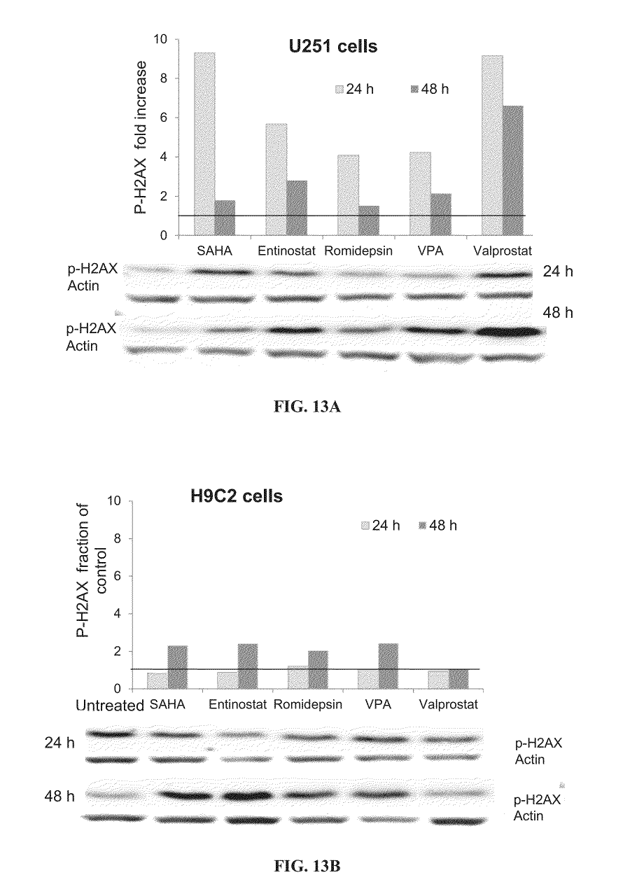

As shown in FIG. 13A, all of the HDAC inhibitors increased levels of pH2AX in U251 cancer cells, with valprostat (AN446) being considerably more effective than the other HDAC inhibitors at increasing pH2AX levels after 48 hours.

In contrast, as shown in FIG. 13B, valprostat did not increase pH2AX levels in cardiomyocytes, whereas all of the other HDAC inhibitors moderately increased pH2AX levels in cardiomyocytes after 48 hours.

These results indicate that AN446 selectively enhances DNA damage to a considerable extent in cancer cells and not in non-cancer cells, whereas other HDAC inhibitors enhance DNA damage in both cancer cells and non-cancer cells.

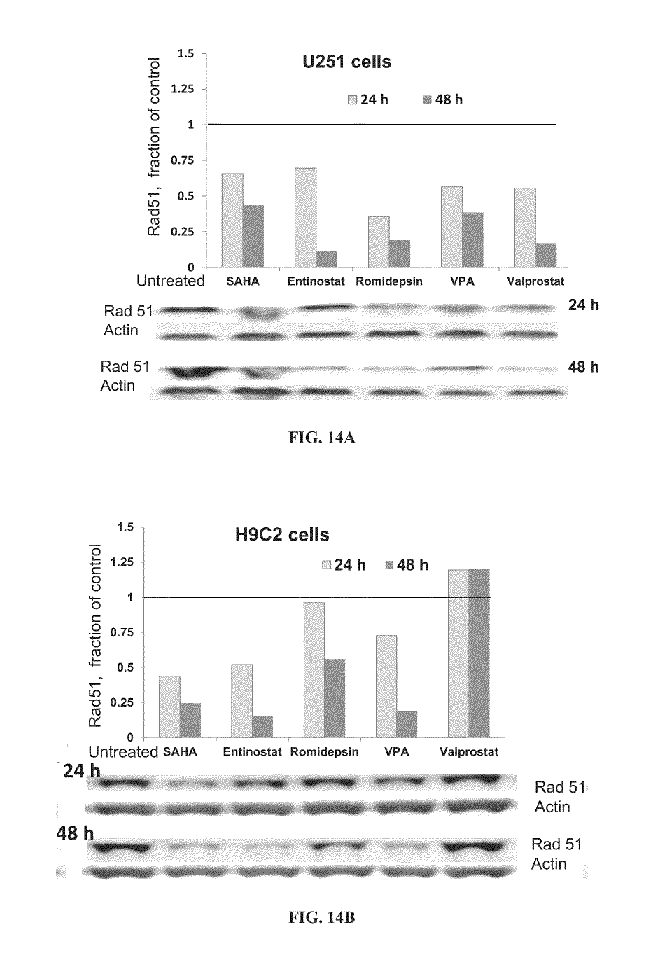

In addition, as shown in FIGS. 14A and 14B, valprostat moderately increased expression of the DNA repair protein Rad51 in cardiomyocytes, whereas all other HDAC inhibitors reduced expression of Rad51 in cardiomyocytes (FIG. 14B); and all of the HDAC inhibitors, including valprostat, reduced expression of Rad51 in U251 cancer cells (FIG. 14A).

These results indicate that AN446 selectively inhibits repair of DNA damage in cancer cells and not in non-cancer cells, whereas other HDAC inhibitors inhibit repair of DNA damage in both cancer cells and non-cancer cells.

These results further confirm that AN446 can selectively target cancer cells, in a manner which is not characteristic of other HDAC inhibitors.

### Example 6

### Comparison of Effects of HDAC Inhibitors on Pro-Inflammatory TLR4 Pathway

TLR4 plays important role in promoting immune escape of cancer cells by inducing production of pro-inflammatory cytokines and apoptosis resistance, contributing to tumor growth and disease-progression.

The effect of AN446 on TLR4 expression in MDA-231 triple negative breast carcinoma cells was compared to the HDAC inhibitors entinostat, romidepsin, SAHA and valproic acid using procedures described in Example 2. The tested compounds were used at concentrations based on the IC₅₀, as described in Example 5.

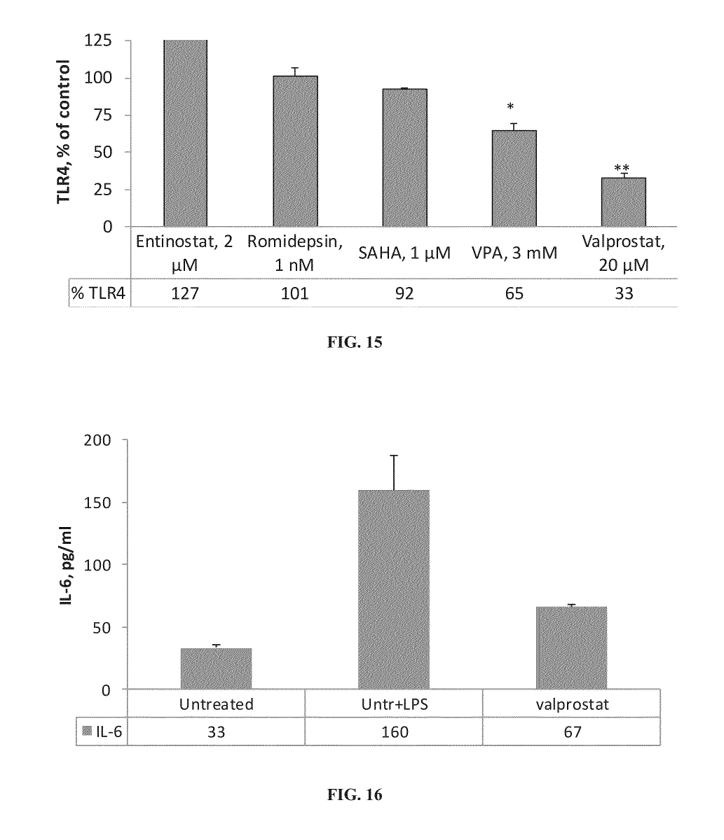

As shown in FIG. 15, valprostat (AN446) was considerably more effective than valproic acid at repressing TLR4 in MDA-231 cells; and SAHA, romidepsin and entinostat had no apparent effect on TLR4 expression.

These results indicate that AN446 can advantageously inhibit TLR4-associated pathways, in a manner in which other HDAC inhibitors do not.

### Example 7

### Effect of AN446 on Inflammation-Associated Pathway in Brain Cells

Increasing evidence suggests that inflammation significantly contributes to the pathogenesis of neurodegenerative diseases. Thus, pro-inflammatory cytokines such as IL-6 influence brain injury and classical neurodegenerative pathways, and contribute to neuronal death [Lozano et al., Neuropsychiatr Dis Treat 2015, 11:97-106; More et al., Mediators Inflamm 2013, 2013:952375]. The ability of AN446 to reduce IL-6 production by human astrocytes was therefore evaluated.

Normal human astrocytes (NHA) were treated with 20 μM AN446, and lipopolysaccharide (1 μg/ml) was then added for 24 hours, and the supernatants of the cultured cells were collected. IL-6 concentrations in cell culture supernatants were quantified by sandwich-ELISA using specific pairs of mAb antibodies (R&D).

As shown in FIG. 16, lipopolysaccharide-induced secretion of IL-6 by human astrocytes was inhibited by valprostat (AN446).

These results indicate that AN446 can protect neuronal tissue such as the brain from neurotoxic processes.

### Example 8

### Effect of HDAC Inhibitors in Combination with Paclitaxel

The interactions between paclitaxel (PXL) and AN446, SAHA or valproic acid were evaluated in MDA-231, T47D and NCI-N87 cells according to the median-effect method, using procedures described hereinabove with respect to Dox.

Paclitaxel has been reported to promote survival of triple negative breast carcinoma (TNBC) cells by activation of TLR4 [Ran, Cancer Res 2015, 75:2405-2410], and TLR4 expression has been reported to correlate with decreased survival, disease progression and metastasis [Yang et al., PLoS ONE 2014, 9:e109980; Bergenfelz et al., Br J Cancer 2015, 113:1234-1243; Mehmeti et al., Breast Cancer Res 2015, 17:130]. In addition, paclitaxel has been reported to upregulate pro-inflammatory mediators such as IL-1β and IL-6 [Pusztai et al., Cytokine 2004, 25:94-102].

As shown in Table 3, paclitaxel (PXL) synergistically interacted with AN446, SAHA and valproic acid in MDA-231 triple negative breast carcinoma cells, and with AN446 in NCI-N87 (gastric carcinoma) cells, whereas PXL did not interact synergistically with any of AN446, SAHA or valproic acid in the T47D non-triple negative breast carcinoma cell line.

These results indicate that the HDAC inhibitors interact synergistically with paclitaxel in cells in overcoming resistance to paclitaxel which is associated with TLR4.

TABLE 3
IC₅₀ values and combination indices (CI)
for drug interactions with paclitaxel

| | Cells | | |
|---|---|---|---|
| Drugs | MDA-231 | T47D | NCI-N87 |
| AN446 (μM) | 37.8 ± 1.1 | 23.6 ± 0.6 | 20 ± 3 |
| AN446 com (μM) | 14.0 ± 0.3 | 13.6 ± 0.6 | 9.1 ± 0.4 |
| Paclitaxel (nM) | 4.4 ± 0.2 | 3.2 ± 0.1 | 4 ± 0.4 |
| Paclitaxel com (nM) | 1.7 ± 0.01 | 2.8 ± 0.1 | 1.8 ± 0.2 |
| CI | 0.8 | 1.4 | 0.76 |
| SAHA (μM) | 1.2 ± 0.3 | 0.6 ± 0.02 | ND |
| SAHA com (μM) | 0.15 ± 0.01 | 0.26 ± 0.02 | ND |
| Paclitaxel (nM) | 4.4 ± 0.2 | 3.2 ± 0.1 | ND |
| Paclitaxel com (nM) | 1.5 ± 0.00 | 2.3 ± 0.01 | ND |
| CI | 0.6 | 1.1 | |
| Valproic acid (mM) | 3.9 ± 0.2 | 4.4 ± 0.3 | ND |
| Valproic acid com (mM) | 1.6 ± 0.01 | 1.8 ± 0.1 | ND |
| Paclitaxel (nM) | 4.4 ± 0.2 | 3.2 ± 0.1 | ND |
| Paclitaxel com (nM) | 1.6 ± 0.01 | 3.6 ± 0.1 | ND |
| CI | 0.8 | 1.1 | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of treating an inflammatory disease or disorder in a subject in need thereof, the method comprising administering to the subject a compound having general Formula I: 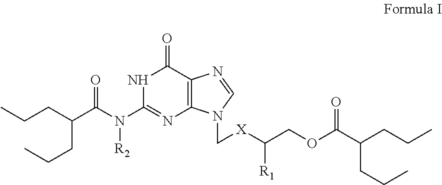
wherein:
X is selected from the group consisting of O and CH₂, or is absent;
R₁ is selected from the group consisting of H, —OR₃, —CH₂OR₃ and —CH₂CH₂OR₃; and
R₂ and R₃ are each independently hydrogen or valproyl,
thereby treating the inflammatory disease or disorder.

2. The method of claim 1, wherein X is O.

3. The method of claim 1, wherein R₁ is selected from the group consisting of H and —CH₂OR₃.

4. The method of claim 1, wherein said disease or disorder is a disease or disorder in which downregulating a protein selected from the group consisting of interleukin-6, MCP-1, TNF-α, and TLR4 is beneficial.

5. The method of claim 4, wherein said disease or disorder is selected from the group consisting of sepsis, cytokine storm, influenza, allergy to nickel, opioid tolerance and/or addiction, hyperalgesia, allodynia, and cancer associated with cancer cells which express TLR4.

6. The method of claim 5, wherein said cancer is selected from the group consisting of triple negative breast carcinoma, gastric carcinoma, lung carcinoma, colon carcinoma, glioblastoma, hepatocellular carcinoma, cervical cancer, endometrial cancer and ovarian cancer.

7. The method of claim 5, wherein disease or disorder is said cancer, and said subject is treated with a taxane drug.

8. The method of claim 1, wherein said disease or disorder is associated with fibrosis.

9. The method of claim 8, wherein said fibrosis is not pulmonary fibrosis or hepatic cirrhosis.

10. The method of claim 8, wherein said fibrosis is of an internal organ.

11. The method of claim 8, wherein said subject is treated with an anticancer therapy.

12. The method of claim 11, wherein said subject is treated with an anticancer drug.

13. The method of claim 12, wherein said anticancer drug is selected from the group consisting of an anthracycline and a platinum-based antineoplastic drug.

14. The method of claim 13, wherein said fibrosis is cardiac fibrosis and said anticancer drug is an anthracycline.

15. The method of claim 13, wherein said fibrosis is renal fibrosis and said anticancer drug is a platinum-based antineoplastic drug.

16. The method of claim 11, further comprising:
administering an anticancer therapy to the subject during a first time period,
following said first time period, determining whether the subject is afflicted with fibrosis, and
administering said compound to a subject determined to be afflicted with fibrosis during a second time period.

17. The method of claim 11, further comprising administering said anticancer therapy to the subject, and co-administering said compound with said anticancer therapy.

18. The method of claim 1, wherein said disease or disorder is an inflammatory neurological disease or disorder.

19. The method of claim 18, wherein said inflammatory neurological disease or disorder is selected from the group consisting of traumatic brain injury, multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis (AML), stroke, an inflammatory retinal disease or disorder, an inflammatory ocular disease or disorder, optic neuritis, spongiform encephalopathy, migraine, headache, cluster headache, and stiff-man syndrome.

20. A method of downregulating a protein selected from the group consisting of interleukin-6, MCP-1, TNF-α, and TLR4 (toll-like receptor 4) in a cell and/or subject, the method comprising contacting the cell and/or administering to the subject an effective amount of a compound having general Formula I: 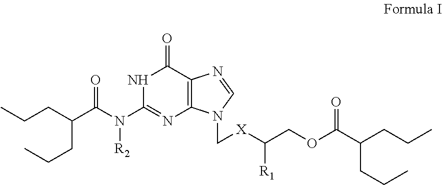
wherein:
X is selected from the group consisting of O and CH₂, or is absent;
R₁ is selected from the group consisting of H, —OR₃, —CH₂OR₃ and —CH₂CH₂OR₃; and
R₂ and R₃ are each independently hydrogen or valproyl,
thereby downregulating the protein.

21. The method of claim 20, wherein X is O.

22. The method of claim 20, wherein R₁ is selected from the group consisting of H and —CH₂OR₃.

23. A method of upregulating interleukin-10 in a cell and/or subject, the method comprising contacting the cell and/or administering to the subject an effective amount of a compound a compound having general Formula I: 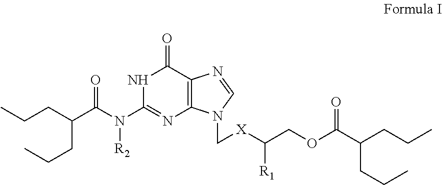
wherein:
X is selected from the group consisting of O and CH₂, or is absent;
R₁ is selected from the group consisting of H, —OR₃, —CH₂OR₃ and —CH₂CH₂OR₃; and
R₂ and R₃ are each independently hydrogen or valproyl,
thereby of upregulating interleukin-10.

24. The method of claim 23, wherein X is O.

25. The method of claim 23, wherein R₁ is selected from the group consisting of H and —CH₂OR₃.


SEQUENCE LISTING (18 sequences)

SEQ ID NO: 1 - DNA, 19 bp, Artificial sequence
  Single strand DNA oligonucleotide
ccaaatcctgtacctcgtc

SEQ ID NO: 2 - DNA, 18 bp, Artificial sequence
  Single strand DNA oligonucleotide
ccacagacaccacatcaa

SEQ ID NO: 3 - DNA, 18 bp, Artificial sequence
  Single strand DNA oligonucleotide
tgaatcgagcagaaccag

SEQ ID NO: 4 - DNA, 20 bp, Artificial sequence
  Single strand DNA oligonucleotide
attctcggcttggatgtgta

SEQ ID NO: 5 - DNA, 18 bp, Artificial sequence
  Single strand DNA oligonucleotide
aaggaagatggacggctg

SEQ ID NO: 6 - DNA, 20 bp, Artificial sequence
  Single strand DNA oligonucleotide
ccaactggagtatttccgtg

SEQ ID NO: 7 - DNA, 17 bp, Artificial sequence
  Single strand DNA oligonucleotide
acagcacccttgtggac

SEQ ID NO: 8 - DNA, 19 bp, Artificial sequence
  Single strand DNA oligonucleotide
gccaatgtctagtccgaat

SEQ ID NO: 9 - DNA, 18 bp, Artificial sequence
  Single strand DNA oligonucleotide
agtccatgccatcactgc

SEQ ID NO: 10 - DNA, 17 bp, Artificial sequence
  Single strand DNA oligonucleotide
accttgcccacagcctt

SEQ ID NO: 11 - DNA, 18 bp, Artificial sequence
  Single strand DNA oligonucleotide
ggcagagaatgctgagtt

SEQ ID NO: 12 - DNA, 18 bp, Artificial sequence
  Single strand DNA oligonucleotide
ctgcatggctggtgtgta

SEQ ID NO: 13 - DNA, 25 bp, Artificial sequence
  Single strand DNA oligonucleotide
tatgaaggaagatggaagattactg

SEQ ID NO: 14 - DNA, 23 bp, Artificial sequence
  Single strand DNA oligonucleotide
atgtgaaatgagattagatgtgg

SEQ ID NO: 15 - DNA, 17 bp, Artificial sequence
  Single strand DNA oligonucleotide
cagcgtcactgtcgatg

SEQ ID NO: 16 - DNA, 15 bp, Artificial sequence
  Single strand DNA oligonucleotide
ccaacgtcgaagccg

SEQ ID NO: 17 - DNA, 22 bp, Artificial sequence
  Single strand DNA oligonucleotide
ctttggtatcgtggaaggactc

SEQ ID NO: 18 - DNA, 22 bp, Artificial sequence
  Single strand DNA oligonucleotide
agtagaggcagggatgatgttc